(19) United States
(12) Patent Application Publication
(10) Pub. No.: US 2019/0217275 A1
(43) Pub. Date: Jul. 18, 2019

(54) CATALYTIC DEPHOSPHORYLATION USING CERIA NANOCRYSTALS

(71) Applicant: THE JOHNS HOPKINS UNIVERSITY, Baltimore, MD (US)

(72) Inventors: Chao Wang, Ellicott City, MD (US); Michael J. Manto, Baltimore, MD (US); Pengfei Xie, Baltimore, MD (US)

(21) Appl. No.: 16/249,730

(22) Filed: Jan. 16, 2019

Related U.S. Application Data
(60) Provisional application No. 62/617,780, filed on Jan. 16, 2018.

(51) Int. Cl. B01J 23/10; C01F 17/00; B01J 23/92; B01J 35/00; B01J 38/68; C07C 201/12

(52) U.S. Cl. CPC B01J 23/10; C01F 17/0043; B01J 23/92; B01J 35/0013; B01J 38/68; C01P 2002/84; C01P 2002/60; C01P 2004/04; C01P 2002/72; C01P 2002/85; C07C 201/12

(57) ABSTRACT

Described are methods of dephosphorylation. Methods of dephosphorylation include contacting a phosphate containing substrate with one or more CeO₂ nanocrystal. Also described is modifying the pH of the dephosphorylation reaction to affect the amount and rate of dephosphorylation. Further described are methods of making CeO₂ nanocrystals of the present disclosure.

### CROSS REFERENCE TO RELATED APPLICATIONS

This application claims priority to U.S. Provisional Application No. 62/617,780, filed on Jan. 16, 2018, the disclosure of which is incorporated herein by reference.

### STATEMENT REGARDING FEDERALLY SPONSORED RESEARCH

This invention was made with government support under contract no. CBET-1437219 awarded by the National Science Foundation. The government has certain rights in the invention.

### FIELD OF THE DISCLOSURE

The disclosure generally relates to reactions catalyzed by nanoparticles and uses thereof for recovery of phosphorus and phosphorus containing-compounds from a variety of sources, including biomass and wastewater.

### BACKGROUND OF THE DISCLOSURE

As an essential element in living systems, phosphorus is crucial for cell division and growth, energy storage and conversion, respiration, photosynthesis, and other biological processes. Phosphorus is found naturally in soil, but concentrations can fall down to very low levels depending on geographic conditions, and artificial phosphorus fertilizers are demanded to sustain the growth of crops. Before the early 1900s, most of the world's phosphorus was derived from animal wastes. Today the vast majority (nearly 80%) of phosphorus in fertilizers comes from phosphate rocks, which are primarily harvested in the remote Western Sahara region. It is predicted that the production of phosphate rocks will reach its peak before 2040 and the reserves will be completely depleted by the end of this century. It thus becomes imperative to develop innovative and sustainable methods for production of phosphorus from renewable sources.

One solution toward this sustainability challenge is to extract phosphorus from phosphorylated biomolecules (e.g., phospholipids and nucleic acids) and recycle it for fertilizer production. This is conventionally done via fermentation, which produces struvite, a precipitate that can be processed to make phosphorus fertilizers. Alternatively, a more robust approach is catalytic dephosphorylation. By hydrolytic cleavage of the phosphate ester bond, free phosphate anions can be released from biomass and agriculture wastes, which can then be captured and regenerated as chemical streams for further applications. Previously studies of cerium oxide (CeO₂) nanocrystals for biological applications (e.g., cancer therapy, pharmacology and toxin mitigation) have shown that these nanomaterials can function as artificial phosphatases and catalyze the dephosphorylation of nucleic acids, peptides, DNA and RNA under ambient conditions. However, very little is known about the catalytic mechanism of CeO₂ during the dephosphorylation reaction of these limited compounds, and it is not known if such nanoparticles could be used in other approaches to recovery of phosphorus recovery. The present disclosure is pertinent to an ongoing need for alternative approaches and compositions for catalytic recovery of phosphorus from, for example, biomass and organic wastes.

### SUMMARY OF THE DISCLOSURE

In embodiments, the present disclosure demonstrates use of CeO₂ nanocrystals (FIG. 1) to demonstrate catalytic recovery of phosphorus using para-nitrophenyl phosphate (p-NPP) as a non-limiting, representative substrate. In certain implementations, the disclosure provides novel performance parameters of CeO₂ for dephosphorylation, including recyclability, activation energies, and turnover frequencies (TOFs).

In various and non-limiting demonstrations, CeO₂ nanocrystals were synthesized with shape control and applied as “artificial phosphatases” to cleave the phosphate ester bond in p-NPP and release free phosphate anions in aqueous solutions. The dephosphorylation reaction was studied on the CeO₂ nanocrystals at various temperatures to evaluate the dependences of rate constant, activation energy and recyclability on the particle shape. The structure-property relationship established as described herein provides evidence that the oxygen vacancies on the surface of CeO₂ are the active sites for dephosphorylation. Furthermore, the present disclosure demonstrates a novel approach to using CeO₂ nanocrystals for sustainable phosphorus production.

In particular embodiments, CeO₂ nanocrystals were synthesized by NaOH-mediated hydrothermal growth with the shape controlled by altering the reaction temperature and/or introducing hexamethylenetetramine (HMT) as a surfactant. Pseudospherical, octahedral, cubic, and rod-like CeO₂ nanocrystals were obtained and subjected to catalytic studies for dephosphorylation in aqueous solutions at temperatures ranging from 5° C. to 95° C. Yields of phosphate and para-nitrophenol (p-NP), rate constants, activation energies and recyclability were systematically evaluated for the CeO₂ nanocrystals of different shapes, which illustrate non-limiting embodiments of the disclosure.

### BRIEF DESCRIPTION OF THE FIGURES

For a fuller understanding of the nature and objects of the disclosure, reference should be made to the following detailed description taken in conjunction with the accompanying figures.

The patent or application file contains at least one drawing executed in color. Copies of this patent or patent application publication with color drawing(s) will be provided by the Office upon request and payment of the necessary fee.

FIG. 1 shows an illustration of the catalytic dephosphorylation of p-NPP.

FIG. 2 shows TEM and HRTEM images of the (a, e) CeO₂ nanospheres, (b, f) CeO₂ nanooctahedra, (c, g) CeO₂ nanorods, and (d, h) CeO₂ nanocubes, respectively. (i) XRD patterns of the CeO₂ nanocrystals indexed to JCDPS No. 65-2795 for CeO₂ with a fluorite type of crystal structure. (j) Ce 3d_{2/3,5/2} XPS spectra of the CeO₂ nanocrystals. Characteristic peaks for Ce³⁺ correspond to the following energy states: u⁰ and v⁰ for Ce(3⁹4f¹)-O(2p⁶) and u¹ and v¹ for Ce(3d⁹4f²)-O(2p⁵). Characteristic peaks for Ce⁴⁺ correspond to the following energy states: u and v for Ce(3d⁹4f²)-O (2p⁴), u^{II} and v^{II} for Ce(3d⁹4f¹)-O(2p⁵), and u^{III} and v^{III} for Ce(3d⁹4f⁰)-O(2p⁶).

FIG. 3 shows (a) UV-Vis spectra for reaction supernatants collected at selected time intervals. The reaction in this sample spectra was catalyzed by CeO₂ nanooctahedra at 25° C. The characteristic absorption peak occurs near 310 nm forp-NPP and near 410 nm for p-NP after pH adjustment. (b) Time-dependent concentration profiles of p-NPP, p-NP and phosphorus determined for dephosphorylation over CeO₂ nanooctahedra at 25° C. Tim-dependent yields of (c)p-NP and (d) phosphorus at 25° C. for each CeO₂ catalyst. (e) Yields of p-NP after 8 h of reaction at various temperatures. Color coding for (c-e) is presented on the right side.

FIG. 4 shows (a) plots of the reaction rate of dephosphorylation depending on time for CeO₂ nanooctahedra at various temperatures. (b, c) Kinetic rate constants (at 25° C.) normalized by mass (kₘ) and surface area (kₛ) of the CeO₂ nanocrystals. (d) Arrhenius plots showing the dependence of rate constant versus temperature and (e) the derived activation energies for the different types of CeO₂ nanocrystals.

FIG. 5 shows (a) O₂-TPD profiles of the CeO₂ nanocrystals with the region for adsorbed oxygen (ad-O₂) zoomed in (b). (c) Correlations of the activation energy and TOF to the surface density of oxygen vacancies estimated based on the amount of ad-O₂ derived from the O₂-TPD analysis.

FIG. 6 shows (a) recyclability of the CeO₂ catalysts measured asp-NP yields from successive runs of the dephosphorylation reaction with each run lasting for 8 h. TEM images of the (b) CeO₂ nanospheres, (c) CeO₂ nanooctahedra, (d) CeO₂ nanorods, (e) CeO₂ nanocubes, and (f) commercial CeO₂ nanocatalyst captured before and after recyclability studies.

FIG. 7 provides compound, graphical and photographic representations related to surface density of oxygen vacancies and turnover frequency.

FIG. 8 shows an HRTEM image of the CeO₂ nanospheres.

FIG. 9 shows an HRTEM image of the CeO₂ nanooctahedra.

FIG. 10 shows an HRTEM image of the CeO₂ nanorods.

FIG. 11 shows an HRTEM image of the CeO₂ nanocubes.

FIG. 12 shows TEM images of the commercial CeO₂ nanocatalyst.

FIG. 13 shows UV-Vis calibration curves for p-NPP at (a) 311 nm and (b) 400 nm, for p-NP at (c) 311 nm and (d) 400 nm, and (e) for phosphate using the molybdenum blue method at 890 nm. The characteristic absorbance peak occurs near 311 nm for p-NPP and near 400 nm for p-NP under alkaline conditions.

FIG. 14 shows yields of p-NP for (a) CeO₂ nanospheres, (b) CeO₂ nanooctahedra, (c) CeO₂ nanorods, (d) CeO₂ nanocubes, and (e) commercial CeO₂ at various temperatures.

FIG. 15 (a) yields of p-NP for CeO₂ nanocubes at various mass loadings, (b) TOF and yields of p-NP as a function of three different loadings of CeO₂ nanocubes.

FIG. 16 shows TEM and HRTEM images of the (a-b) CeO₂ nanospheres and (c-d) CeO₂ nano-octahedra, respectively. (e) XRD patterns of the CeO₂ nanocrystals indexed to JCDPS no. 65-2795 for the fluorite crystal structure of CeO₂. (f) Ce 3d_{2/3,5/2} XPS spectra of the CeO₂ nanocrystals. Characteristic peaks for Ce³⁺ correspond to the following energy states: uand v⁰ for Ce(3d⁹4f¹)-O(2p⁶) and u^{I} and v^{I} for Ce(3d⁹4f²)-O(2p⁵). Characteristic peaks for Ce⁴⁻ correspond to the following energy states: u and v for Ce(3d⁹4f²)-O (₂p⁴) u^{II} and v^{II} for Ce(3d⁹4f¹)-O(2p⁵), and u^{III} and v^{III} for Ce(3d⁹4f⁰)-O(2p⁶). (g) O₂-TPD profiles of the CeO₂ nanocrystals with zoomed-in detail on the region for adsorbed oxygen (ad-O₂) (inset).

FIG. 17 shows yields of (a and d) P and (b and e)p-NP after 8 h (hour) of reaction at various temperatures for CeO₂ nanospheres and nano-octahedra, respectively. Arrhenius plots for (c) CeO₂ nanospheres and (f) CeO₂ nano-octahedra.

FIG. 18 shows correlations of (a) yield of P at 25° C. after 8 h of reaction time, (b) TOF at 25° C. and (c) activation energy to reaction solution pH.

FIG. 19 shows (a) adsorption equilibrium constants and (b) percentage of oxygen vacancies occupied by phosphate as a function of pH. (c) Turnover frequency and (d) activation energy as a function of occupied surface oxygen vacancies.

FIG. 20 shows (Left) a schematic illustration of the solution pH effect on catalytic dephosphorylation using CeO₂ nanocrystals. The surface oxygen vacancy is identified as the active sites, on which the coverage of phosphate anions is subject to pH. (Right) For both nano-octahedra and nanospheres, the activation energy of the dephosphorylation reaction increases as the occupancy of phosphate adsorbates increases, suggesting a relative high pH (e.g., 8-11) is beneficial for the reaction by mitigating phosphate adsorption and reducing the activation energy for the dephosphorylation reaction.

FIG. 21 shows a table showing volumes of acidic and basic pH modifier solutions added to the 10 mL p-NPP reaction solutions for each tested pH value.

FIG. 22 shows a table showing volumes of acidic and basic pH modifier solutions added to the 10 mL p-NPP reaction solutions for each tested pH value.

FIG. 23 shows TEM images after exposure to acidic and alkaline reaction solutions. TEM images of the CeO₂ (a-b) nanospheres and (c-d) nano-octahedra after 8 h of exposure to reaction mixtures with pH values of 1 and 11, respectively.

FIG. 24 shows phosphate adsorption profiles. Percent of vacancies occupied by phosphate as a function of time for the nanospheres and nano-octahedra at solution pH values of 1 and 11.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

All ranges provided herein include all values that fall within the ranges to the tenth decimal place, unless indicated otherwise. The disclosure includes all kinetic parameters and ranges thereof, all amounts of CeO₂ nanocrystals and ratios thereof to phosphorus containing substrates, all temperatures, all time parameters, all volumes, all densities of oxygen densities on the CeO₂ nanocrystals, all oxygen concentrations, a nanoparticle actual dimensions, average dimensions, and size distributions, and systems comprising the CeO₂ nanocrystals, kits containing the CeO₂ nanocrystals, all methods and processes described herein, including but not limited to methods of separating and recovering phosphorus (such as in the form of a phosphate) from substrates.

Embodiments of the disclosure includes complexes of the CeO₂ nanocrystals and phosphorus containing substrates. In embodiments, the disclosure includes a reaction comprising CeO₂ nanocrystals and a phosphate containing substrate wherein the CeO₂ nanocrystals catalyze cleavage of a covalent bond comprising a phosphate, such as cleavage of a phosphate ester. In embodiments, the disclosure includes catalyzing a dephosphorylation reaction by: a) contacting a CeO₂ nanocrystal with a phosphate containing substrate to form a reaction mixture; and b) incubating the reaction mixture reaction mixture for a period of amount of time; wherein after the period of time, the phosphate containing substrate is converted into a dephosphorylated product. As described further below, the disclosure includes separating a phosphorus and/or a phosphorus containing compound, such as a phosphate, from the dephosphorylated product.

The pH of the reaction mixture may affect dephosphorylation. In embodiments, desirable pH values are 1-11 (e.g., 1, 2, 3, 4, 5, 6, 7, 8, 9, 10, or 11), including every 0.1 pH value and range therebetween. In embodiments, the pH is 8-11 (e.g., 8, 9, 10, or 11), including every 0.1 pH value and range therebetween. Without intending to be bound by any particular theory, it is considered that high pH (e.g., 8-11) may result in desirable dephosphorylation.

In embodiments, the disclosure provides a recovery device for collecting and recovering phosphorus and/or phosphorus-containing compounds, such as phosphate, the device including CeO₂ nanocrystals described herein. Thus, the disclosure includes an apparatus that is useful in phosphorus-stripping applications, which may comprise, for example, a column or other device into which the CeO₂ nanocrystals are incorporated, such as a filter, a membrane, a container, or any other suitable apparatus.

In non-limiting embodiments, the phosphorus containing substrates can be present in, for example, any biomass, e.g., any organic material that comes from plants matter and/or animals, raw sewage, or a sludge feed from sewage treatment facility, or any effluent that contains a fluid from which the removal of phosphorus would be desirable (e.g., wastewater effluent). In embodiments, the disclosure provides a method for phosphorus/phosphate removal and recovery from a main wastewater process stream, or any liquid waste streams, such as from biologically treated wastewater. In embodiments, the disclosure provides re-circulation of water over nanoparticles described herein more than one time to enhance phosphorous recovery.

Aspects of this disclosure can be integrated into any known wastewater treatment methods and devices and systems, including but not limited to protein-based enzymatic treatments, biological treatments (e.g., bacteria-based treatments), and chemical treatments. In embodiments, a method of this disclosure comprises exposing a liquid stream comprising phosphorus to the CeO₂ nanocrystals that are described herein. The liquid stream may come from various sources, such as from wastewater treatment, mining processes or other industrial processes. The liquid stream may be pre-treated to reduce organic compounds or solids. Thus, in certain approaches, a method of this disclosure can be performed during or after conventional biological treatment of wastewater, where organic compounds and/or suspended solids removal is also performed concurrently or sequentially with the catalytic dephosphorylation using the CeO₂ nanocrystals that are described herein.

In embodiments, the disclosure thus provides an apparatus for treatment of a solution or suspension or other liquid comprising phosphorus-containing substrates. The apparatus generally comprises a container comprising CeO₂ nanocrystals as described herein (e.g., CeO₂ nanocrystals present in a solution or affixed to a substrate in the apparatus), and at least an inlet through which the solution can pass, wherein the phosphorus-containing substrates are allowed to contact the CeO₂ nanocrystals such that phosphorus is separated from the substrate, and wherein phosphorus separated from the substrates can be recovered, and if desired, purified to any desired degree of purity, concentration, etc., for use in any of a wide variety of applications, including but not limited to fertilizer production. Thus, recovery of the phosphorus can be achieved using any suitable approach, many of which are known in the art and can be adapted for use in the present compositions and methods, given the benefit of this disclosure.

Thus, methods of this disclosure generally comprise contacting a liquid (or gaseous) sample comprising phosphorus that is a component of a substrate with the CeO₂ nanocrystals such that the phosphorus is separated from the substrate, and optionally recovering the separated phosphorus. The CeO₂ nanocrystals can be in contact with the substrate for any suitable period of time, ranging from seconds, to minutes, to hours, or days. The reaction temperatures can be varied, and can range from ambient temperature (i.e., from about 20 to 25° C.), or can be performed at a reduced temperature, such as 3° C., or an elevated temperature, such as up to 95° C., or higher. In embodiments, a method is performed at from 5 to 95° C., inclusive, and including all ranges of integers there between. In an embodiment, a method is performed at a temperature of from 5-65° C.

In embodiments, the contacting a CeO₂ nanocrystal with a phosphate containing substrate to form a reaction mixture further comprises i) adjusting the reaction mixture before incubating to a first temperature (e.g., 3 to 95° C., including all 0.1° C. values and ranges therebetween); and ii) allowing the reaction mixture to remain at the first temperature for a first amount of time (e.g., 1 second to 8 hours, including every 0.1 second value and range therebetween); where after the first amount of time, the phosphate containing substrate is converted into a dephosphorylated product.

In embodiments, the CeO₂ nanoparticles are provided as any one, or as a mixture of nanospheres, nanooctahedra, nanocubes, and nanorods. All sizes and ranges of sizes of nanoparticles, and size distributions, described herein are encompassed, non-limiting examples of which are provided in Table 1, such ranges being variable by at least +/− 10%. In certain non-limiting embodiments, the CeO₂ nanospheres have an average diameter of 4 nm, CeO₂ nanooctahedra have a size of ˜18 nm, CeO₂ nanorods have a diameter of 10 nm on average, with a length varying from 50 to 150 nm, CeO₂ nanocubes have a distribution of particle size, with an edge length varying from 20 to 120 nm. The CeO₂ nanoparticles can exhibit any range of crystallinity that is described herein.

In embodiments, the CeO₂ catalyst can be removed by any suitable approach, such as by centrifugation, and can be used in the presence of any suitable solvent, which can be removed using standard techniques, including but not limited to evaporation. In embodiments, the pH of the reaction can be altered by adapting a wide variety of approaches and reagents which will be apparent to those skilled in the art given the benefit of the present disclosure. The degree, amount or any other measurement of the recovery of phosphorous or phosphorus-containing compound can be determined using approaches that are well known to those skilled in the art. The disclosure includes recovery of some, essentially all, or all of the phosphorus-containing compound that comes into contact with the CeO₂ nanocrystals that are described herein. In embodiments, at least 50%-99% of the phosphorus-containing compound is recovered. In embodiments, recovery of the phosphorus-containing compound comprises a measurement of desorption of phosphate from the catalyst surface.

In embodiments, the method of the present disclosure further comprises i) centrifuging the reaction mixture after incubating and forming a supernatant and a pellet; ii) removing the supernatant; iii) suspending the pellet in a solution; and iv) sonicating the solution and pellet such that the pellet re-disperses into the solution.

In embodiments, the recovery of phosphorous or any phosphorus-containing compound such as phosphate using the compositions and methods of this disclosure can be compared to a suitable control, wherein the recovery of the phosphorus-containing compound achieved by using an approach of this disclosure exhibits at least one of: greater recover relative to the control, faster recovery related to the control, less expensive recovery related to a control, or more pure phosphorous or any phosphorus-containing relative to a control. The control can be any suitable value, including but not necessarily limited to recovery of phosphorus or and/or any phosphorus-containing compound, such as phosphate, that is achieved using, for example, a dephosphorylase biological enzyme.

In embodiments, a catalyst of the present disclosure can be recycled. Recycling a catalyst of the present disclosure comprises: i) suspending a used catalyst comprising CeO₂ nanocrystals in a solution; and ii) modulating (e.g., sonicating) the solution and the used catalyst such that the used catalyst re-disperses into the solution. The method may optionally further comprise incorporating the phosphorous and/or the phosphorus-containing compound from the dephosphorylated product into a distinct product.

The recovered phosphorous may be in various forms of phosphorous which will be recognized by those skilled in the art, including but not limited to free phosphate anions, such as those in an aqueous solution, or as an acid. In one embodiment, phosphorous that is liberated from the substrate via the activity of the CeO₂ nanocrystals that are described herein is precipitated.

The disclosure includes using the recovered phosphorus and/or phosphorous containing compounds to make any phosphorus containing product, and such products made using the recovered phosphorus containing products are also included. In a non-limiting embodiment phosphorous is separated from a substrate as described herein, and is incorporated into a fertilizer product, a detergent, a surfactant, a plastic, a lubricant, a food additive or supplement, pesticides, various phosphorus containing acids that are used in numerous industrial processes, such as phosphoric acid, as well as phosphorus chlorides, phosphorus sulfides, elemental phosphorus, red or yellow phosphorus, or any other phosphorus/phosphate containing product.

In more detail, the present disclosure provides for use of CeO₂ nanocrystals

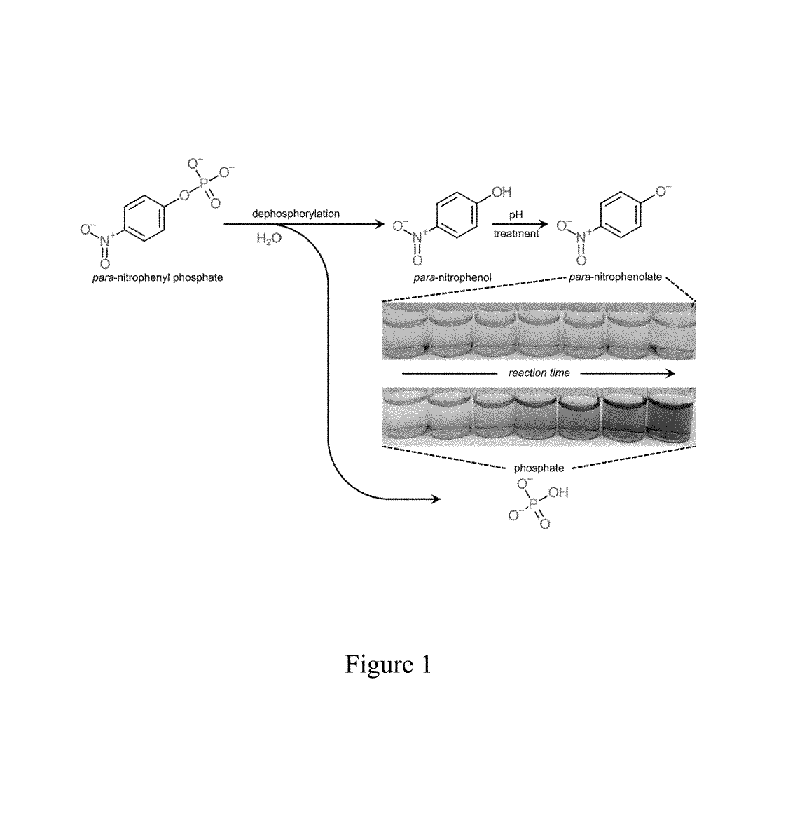

(FIG. 1) to provide for catalytic recovery of phosphorus, wherein the CeO₂ nanocrystals essentially function as artificial enzymes that can catalyze dephosphorylation of phosphorous containing substrates. The method is demonstrated using para-nitrophenyl phosphate (p-NPP) as a non-limiting model substrate. In certain implementations, the disclosure reveals novel performance parameters of CeO₂ for such dephosphorylation purposes, which include but are not limited to recyclability, activation energies, and turnover frequencies (TOFs).

In various and non-limiting demonstrations, CeO₂ nanocrystals were synthesized with shape control and applied as artificial phosphatases to cleave the phosphate ester bond in p-NPP and release free phosphate anions in aqueous solutions. The dephosphorylation reaction was studied at various temperatures to evaluate the dependences of rate constant, activation energy and recyclability on the particle shape. The structure-property relationship established as described herein provides evidence that the oxygen vacancies on the surface of CeO₂ are the active sites for dephosphorylation. Thus, and without intending to be bound by any particular theory, it is considered that the present disclosure demonstrates a novel approach to using CeO₂ nanocrystals for sustainable phosphorus production in a wide variety of industries.

In particular embodiments, CeO₂ nanocrystals were synthesized by NaOH-mediated hydrothermal growth with shape of the nanocrystals controlled by altering the reaction temperature and/or introducing hexamethylenetetramine (HMT) as a non-limiting example of a surfactant. Pseudospherical, octahedral, cubic, and rod-like CeO₂ nanocrystals were obtained and subjected to catalytic analysis for dephosphorylation in aqueous solutions at temperatures ranging from 5° C. to 95° C. Yields of phosphate and para-nitrophenol (p-NP), rate constants, activation energies and recyclability were systematically evaluated for the CeO₂ nanocrystals of different shapes, which illustrate non-limiting embodiments of the disclosure. The disclosure is therefore considered to be expandable for treating a wide-variety of phosphorus-containing substrates, and for producing phosphorous for use in vast applications as described above.

The present disclosure also describes kits comprising CeO₂ nanocrystals of the present disclosure. Such a kit may comprise printed material including instructions for using the CeO₂ nanocrystals to separate phosphorus or a phosphorus containing compound from a substrate.

The following examples are presented to illustrate the present disclosure. They are not intended to limiting in any matter.

### EXAMPLE 1

This example provides a description of dephosphorylation catalyzed by ceria nanoparticles (e.g., nanocrystals).

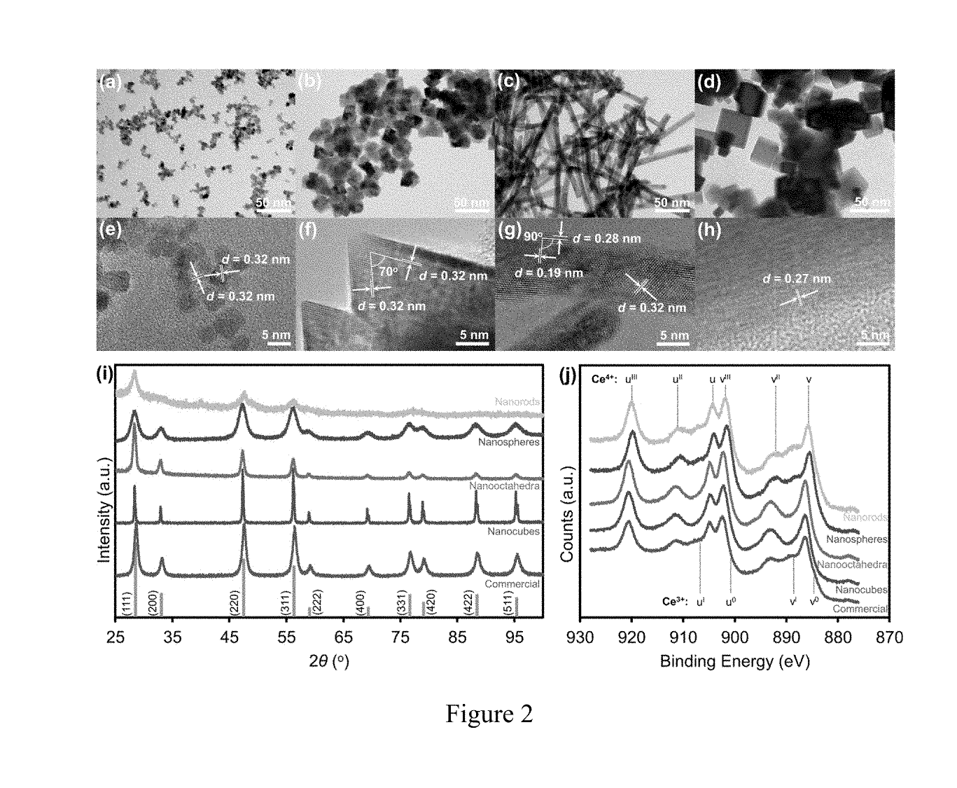

Synthesis and Characterization of CeO₂ Nanocrystals. FIG. 2(a-h) shows the TEM and HRTEM images of the as-synthesized CeO₂ nanocrystals of different morphologies.

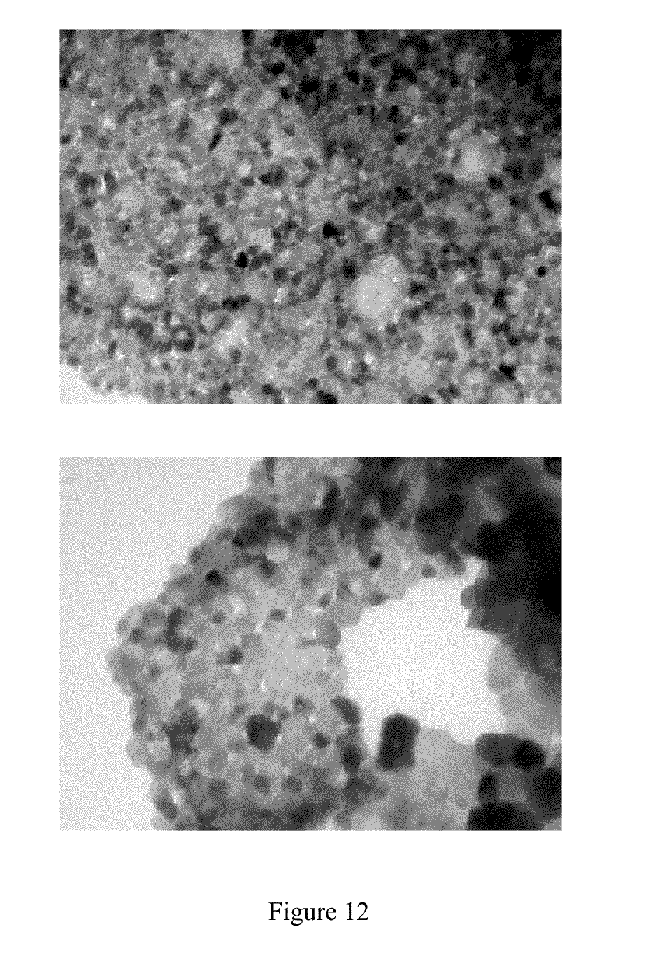

The CeO₂ nanospheres have an average diameter of 4 nm (FIG. 2(a)). Although the lattice fringes exhibited in the HRTEM images (FIG. 2(e)) can be ascribed to (111) planes (with an inter-plane spacing of 0.32 nm) of CeO₂ in the fluorite phase, the CeO₂ nanospheres have no preferential exposure of a certain facet on the surface. The CeO₂ nanooctahedra have a size of ˜18 nm with (111) facet preferentially exposed on the surface (FIG. 2(b, f)). The CeO₂ nanorods have a diameter of 10 nm on average, with the length varying from 50 to 150 nm (FIG. 2(c)). It can be seen from the HRTEM images that the CeO₂ nanorods possess a polycrystalline nature, likely with (100) and (110) facets (with inter-plane distances of 0.19 nm and 0.28 nm, respectively) preferentially exposed on the sidewalls (FIG. 2(g)). The CeO₂ nanocubes have a rather wide distribution of particle size, with the edge length varying from 20 to 120 nm, and the surface is dominated by (100) facets (FIG. 2(d, h)). FIG. 2(i) presents the XRD patterns collected for the CeO₂ nanocrystals, in comparison to commercial CeO₂ nanopowders (see FIG. 12). The major peaks at 28.5°, 33.1°, 47.5° , and 56.3° can be assigned to the (111), (200), (220), and (311) planes of CeO₂ adopting the fluorite structure (space group Fm3̅m, JCDPS No. 65-2795). Among the nanocrystals of different shapes, CeO₂ nanorods exhibit lower peak intensities, indicating poorer crystallinity than the others, which is consistent with the observations from HRTEM images. A summary of the average particle sizes determined from TEM images and the specific surface areas measured by BET analysis is presented in Table 1.

It has been reported that oxygen vacancies are usually present in CeO₂ nanocrystals, which are associated with Ce³⁺ and can be characterized by XPS. FIG. 2(j) shows the Ce 3d XPS spectra collected for the CeO₂ nanocrystals. The spectra exhibit two multiplets (denoted as v and u) that correspond to the 3d_{5/2} and 3d_{3/2} core holes of Ce and have a spin-orbit splitting of ˜18.6 eV. All the peaks in the spectra for octahedral and cubic nanocrystals can be ascribed to Ce⁽⁴⁺⁾ with each multiplet comprised of three peaks and a total of six peaks assigned to three different energy states: u (904 eV) and v (886 eV) for Ce(3d⁹4f²)-O(2p⁴), u^{II} (911 eV) and v^{II} (893 eV) for Ce(3d⁹4f¹)-O(2p⁵), and u^{III} (920 eV) and v^{III} (902 eV) for Ce(3d⁹4f⁰)-O(2p⁶). For nanorods and nanospheres, four additional peaks are present in the spectra, which are believed to be associated with Ce³⁺: u⁰ (901 eV) and v⁰ (884 eV) for Ce(3d⁹4f¹)-O(2p⁶), and u^{I} (907 eV) and v^{I} (889 eV) for Ce(3d⁹4f²)-O(2p⁵). Stronger Ce³⁺ peaks were observed for the CeO₂ nanorods and nanospheres than for the cubic and octahedral nanocrystals. Without intending to be bound by any particular theory, this could correspond to the presence of more oxygen vacancies on the former two types of nanocrystals, considering the formation energy of oxygen vacancy follows the trend (110)<(100)<(111) for the various low-index facets of CeO₂. This correlation is adjustable by the potential presence of impurity phases (such as Ce₂O₃) in small amounts and by the observation that the probing of Ce³⁺ by XPS is not surface-specific for the CeO₂ nanocrystals.

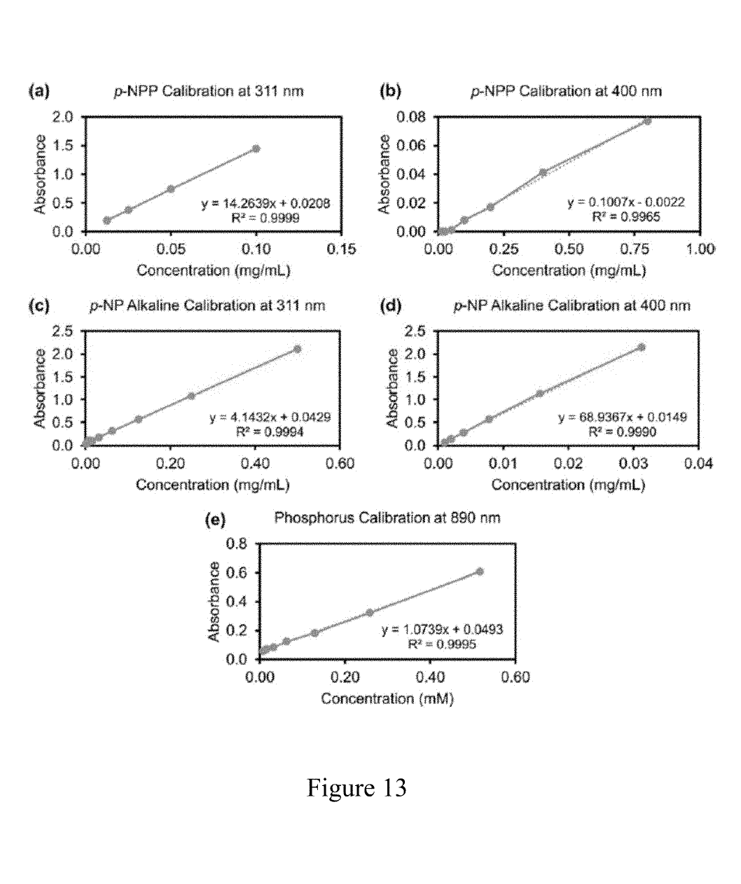

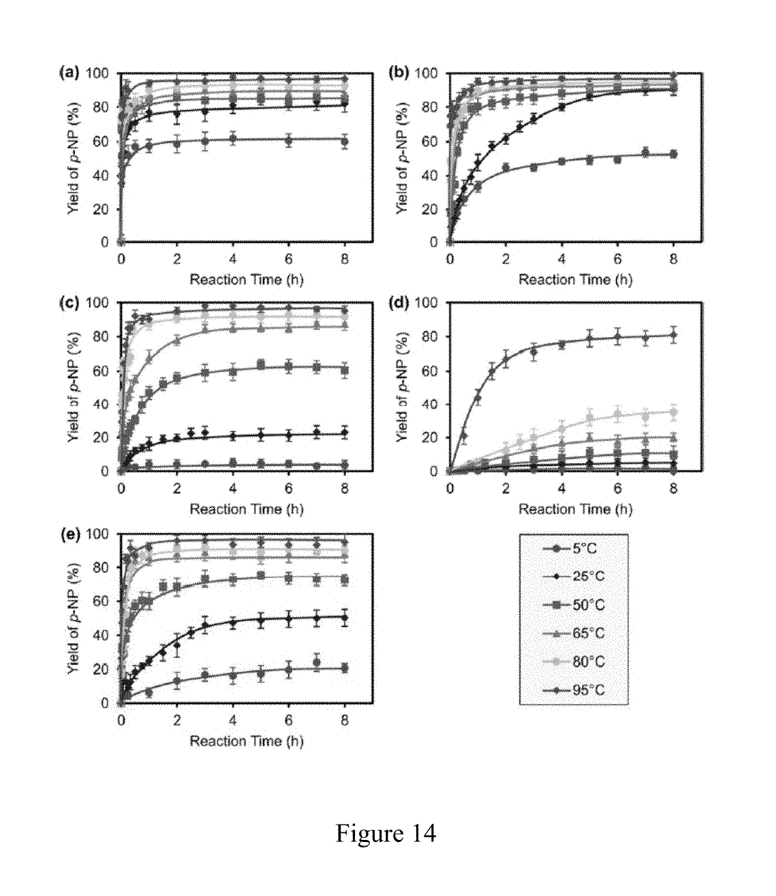

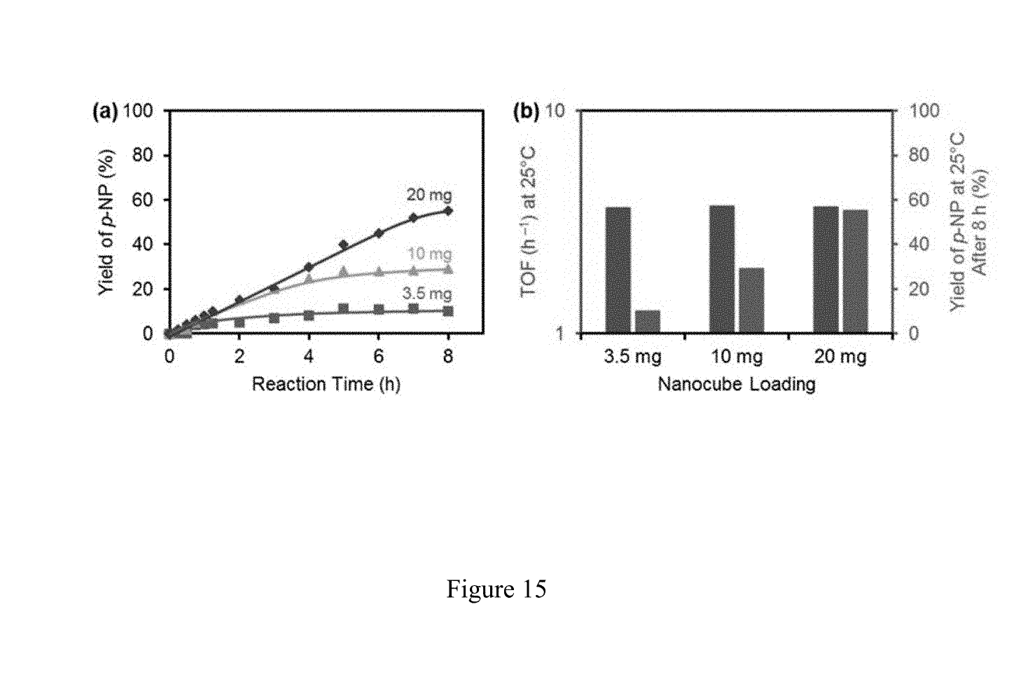

Catalytic Studies and Kinetics of Dephosphorylation. The CeO₂ nanocrystals of different shapes were applied as catalysts for dephosphorylation of para-nitrophenyl phosphate (p-NPP) in aqueous solutions. p-NPP is a common chromogenic substrate used in enzyme-linked immunosorbent assay (ELISA) and spectrophotometric analysis of phosphatases. Both p-NPP and its hydrolysis product, p-NP (converted into para-nitrophenolate after the pH adjustment, exhibit distinct optical absorption properties, making it possible to track the reaction progress by using spectroscopic means. Meanwhile, the produced phosphate can be analyzed by using the known molybdenum blue assay. The concentrations of p-NPP, p-NP and phosphate can thus be determined by comparing against the corresponding standard solutions (FIG. 13).

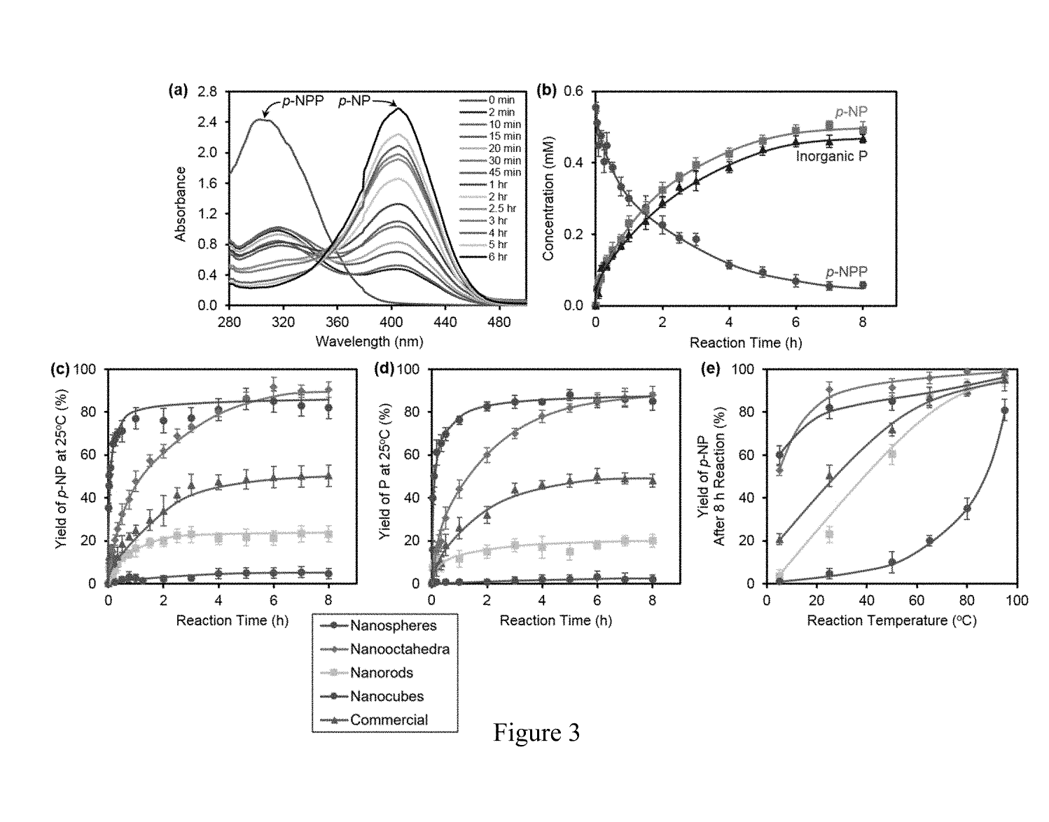

FIG. 3(a) shows the UV-Vis spectra collected over the course of dephosphorylation of p-NPP on CeO₂ nanooctahedra at 25° C. The peak corresponding top-NPP near 310 nm dissipated as the reaction proceeded, accompanied with the gradual increase of peak intensity for p-NP near 405 nm, indicating the conversion of p-NPP top-NP. FIG. 3(b) summarizes the concentrations of p-NPP, p-NP and phosphate depending on the reaction time. It can be seen that the conversion of p-NPP top-NP and phosphate was nearly stoichiometric, indicating that no undesirable side reactions or competing products were involved in the dephosphorylation process.

The CeO₂ nanocrystals of different shapes had substantially different performances in the dephosphorylation of p-NPP. FIG. 3(c) shows the comparisons of time-dependent reaction yields among the various types of CeO₂ nanocrystals at 25° C. The nanospheres and nanooctahedra delivered much higher yields of p-NP and phosphate than the nanocubes and nanorods. After 8 h of reaction, the yield of p-NP reached 82.0±5.0%, 90.5±3.6%, 82.0±5.0%, 23.0±3.8%, and 4.7±2.5% for the nanospheres, nanooctahedra, nanorods, and nanocubes, respectively. Similar trends were also observed in the yield of phosphate (FIG. 3(d)). It is noteworthy that the yields of phosphate were consistent with the yields of p-NP for the different types of CeO₂ nanocrystals, suggesting the complete desorption of phosphate from the catalyst surface. Besides changing with reaction time, the reaction yields were also found to be dependent on the temperature (FIG. 3(e)). For all the CeO₂ nanocrystals, higher yields were observed as the reaction temperature increased, reaching ˜90% after 8 h at 95° C. In the temperature range of 5-65° C., nanospheres and nanooctahedra outperformed the nanocubes and nanorods.

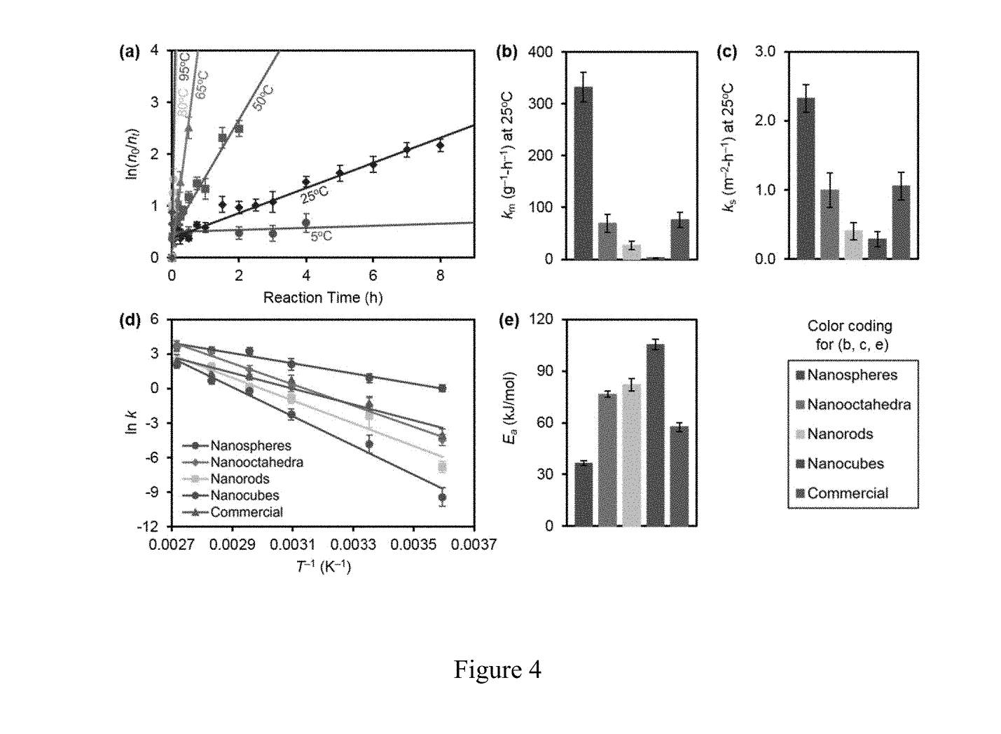

FIG. 4 summarizes the results of kinetic analysis for the various types of CeO₂ nanocrystals. The dephosphorylation of p-NPP was found to be first order with respect top-NPP (FIG. 4(a)). For nanooctahedra, the rate constants were measured to be 0.011±0.001, 0.24±0.06, 1.1±0.4, 5.0±0.5, 25±2 and 39±2⁻¹ at 5° C., 25° C., 50° C., 65° C., 80° C. and 95° C., respectively. Rate constants normalized by catalyst loading (kₘ, FIG. 4(b)) and specific surface area (kₛ, FIG. 4(c)) for the CeO₂ nanocrystals of different shapes follows the trend: nanosphere >nanooctahedra>nanorod>nanocube. The most active nanospheres had a kₘ value of 331±29 g⁻¹h⁻¹ and kₛ of 2.3±0.2 m⁻² h⁻¹. The increase of rate constant with temperature indicates a positive value of the apparent activation energy, as seen from the Arrhenius plots for the various CeO₂ catalysts presented in FIG. 4(d). The activation energy was found to be 36.6±1.2 kJ/mol, 76.5±1.9 kJ/mol, 82.0±3.6 kJ/mol, and 105.4±2.9 kJ/mol for the nanospheres, nanooctahedra, nanorods, and nanocubes, respectively, in comparison to 57.4±2.7 kJ/mol for the commercial CeO₂ catalyst (FIG. 4(e)). The trend of activation energy correlates inversely to that for TOF, highlighting the structure sensitivity of the dephosphorylation reaction on CeO₂.

Active Sites on CeO₂ Catalysts. From above it can be seen that the catalytic activity for dephosphorylation is dependent on the shape of the CeO₂ nanocrystals. The catalytic activity follows the order: nanospheres>nanooctahedra>nanorods>nanocubes, whereas the trend for activation energy is inversed. Considering the preferential exposure of (111) on the nanooctahedra, (110) and (100) on the nanorods and (100) on the nanocubes, it is suggested that the catalytic activity for dephosphorylation of the different low-index facets of CeO₂ follows the trend: (111)>(110)>(100). This dependence could be correlated to the Lewis acidity of the surface Ce⁴⁺ cations, which can coordinate phosphoryl oxygen and activate the dissociation of the P—O bond. It is however noticed that the correlation between surface structures and acid-base properties of CeO₂ facets is still under debate in the literature. It has been believed that the acidity of Ce⁴⁺ on ordered, defect-free surfaces follows the order (100)>(110)>(111), but this trend is obviously opposite to and cannot explain the observed dependence of dephosphorylation catalytic activity on the shape of CeO₂ nanocrystals. Meanwhile, it is noticed that spectroscopic investigations on CeO₂ nanocrystals show weak dependence of acidity on the nanocrystal shape and surface facets.

Besides Ce⁴⁺ on the ordered facets, the XPS analysis has shown the presence of Ce³⁺ in the CeO₂ nanospheres and nanorods (FIG. 2(j)). It is reported that surface Ce³⁺ sites could play an important role in the dephosphorylation reaction considering the biomimetic functionality of binuclear Ce(III)-Ce(IV) complex. As Ce³⁺ is usually associated with the formation of oxygen vacancy, whereas the formation energy of vacancy varies among the different facets of CeO₂, it is plausible that the dependence of catalytic activity on the particle shape is a result of the different surface densities of oxygen vacancy. Studies of temperature-programmed desorption of oxygen (O₂-TPD) was thus performed for quantitative analysis of the oxygen vacancies on the surface of the CeO₂ nanocrystals.

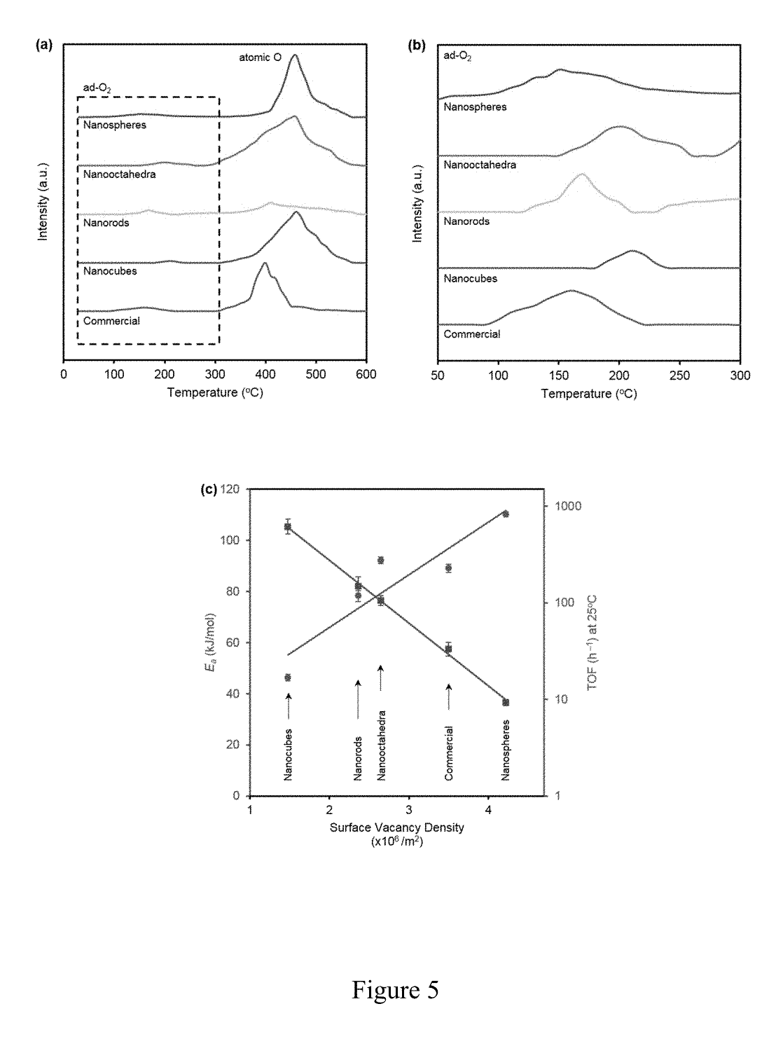

FIG. 5(a, b) presents the patterns for temperature-programmed desorption of oxygen (O₂-TPD) recorded on the various types of CeO₂ nanocrystals. Two desorption peaks are observed in these profiles: the first one at 150-210° C., which can be assigned to adsorbed molecular oxygen (ad-O₂) on oxygen vacancies, and the second one at 400-460° C. corresponding to atomic oxygen evolved from the bulk of CeO₂. Surface density of oxygen vacancy estimated based on the amounts of ad-O₂ follows the order: nanospheres>commercial >nanooctahedra nanorods≅nanocubes (FIG. 5(c)). This trend correlates well to the dependence of activation energy on the particle shape, namely decreasing Eₐ with increasing surface density of oxygen vacancies. Moreover, the turnover frequency (TOF) derived from normalization of the rate constant with the surface density of oxygen vacancies was found to increase as the surface vacancy density increases, reaching 827±46 h⁻¹ for the most active nanospheres. These findings indicate that the active sites for dephosphorylation on CeO₂ are associated with the oxygen vacancies, with the surface density of oxygen vacancies being a good descriptor for the reaction kinetics. The catalytic mechanisms associated with oxygen vacancies are thus believed to involve activation of the phosphoryl ester molecules at acid-base pair centers such as Ce³⁺-O, □-O (□ denotes an oxygen vacancy site), or □-OH (hydroxyl group adsorbed on nearby Ce cation sites). It is further noted that the onset temperature for oxygen desorption follows a similar trend as the surface density of oxygen vacancy, with the nanospheres having the lowest onset temperature (FIG. 5b). This finding indicates that the nanospheres have the weakest binding to ad-O₂, which may also facilitate the desorption of dephosphorylation products (phosphate and/or p-NP).

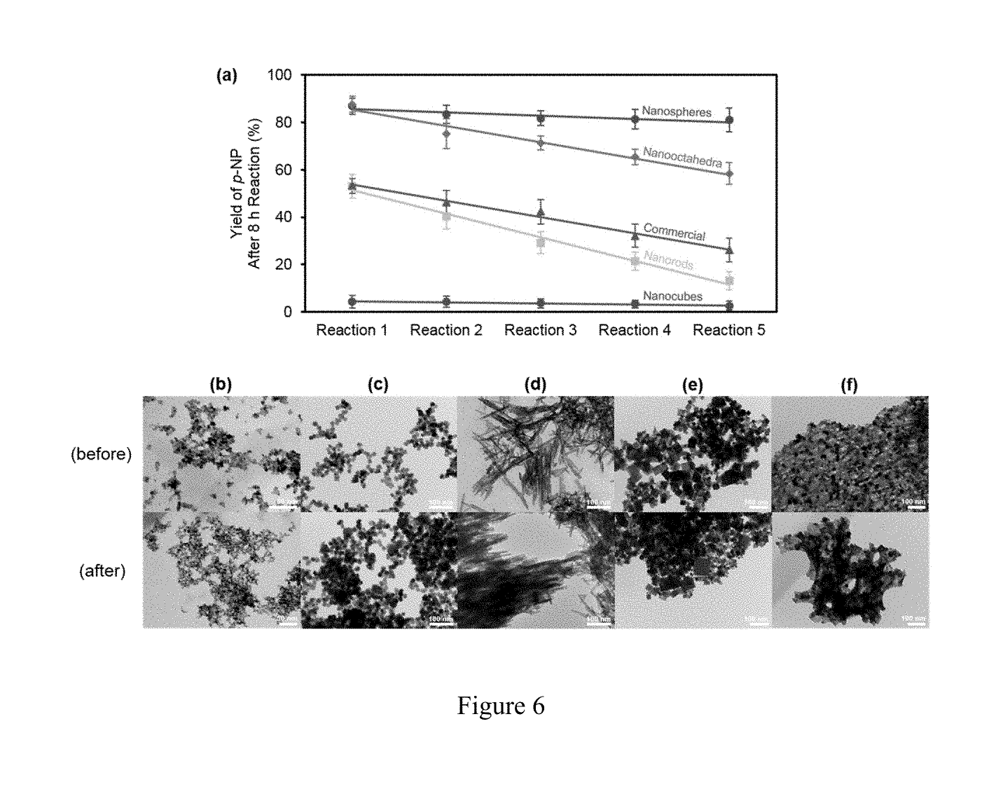

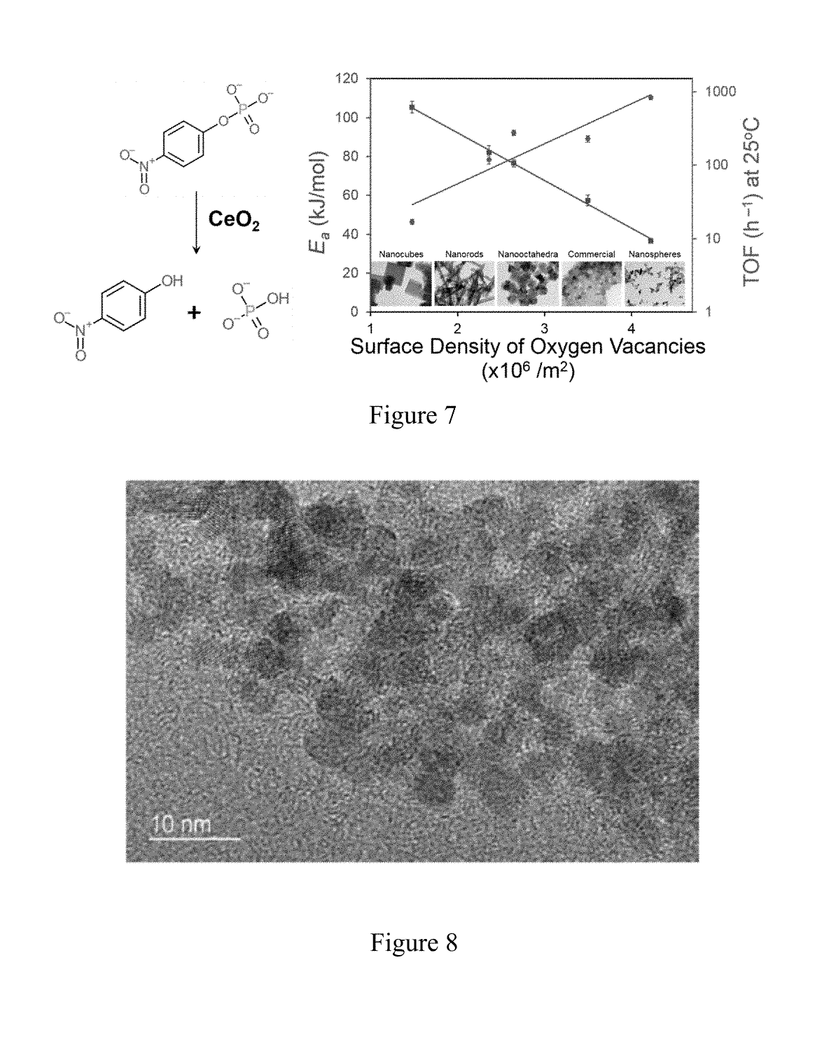

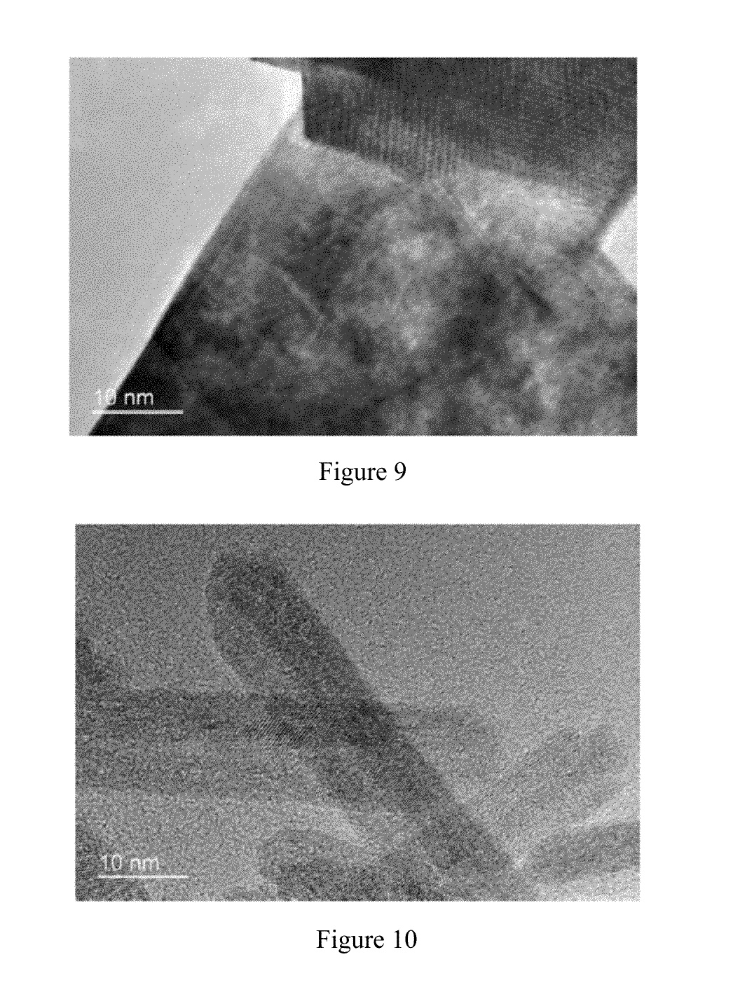

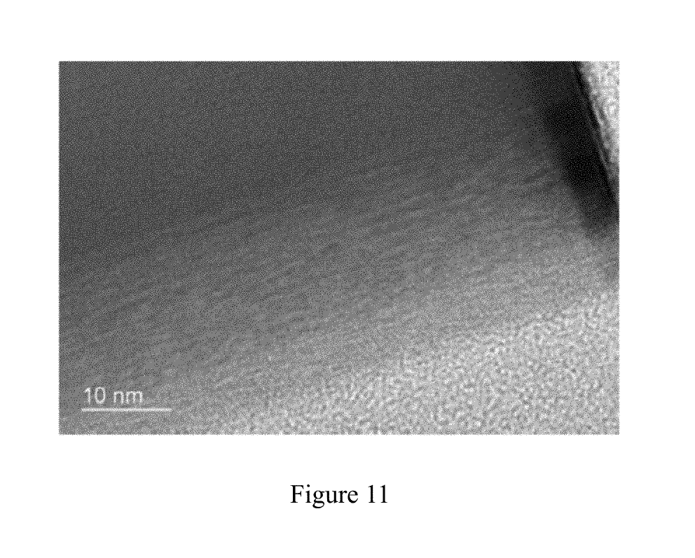

Catalyst Recyclability. Recyclability represents an important merit of catalysts in practical applications. FIG. 6 summarizes the evolution of p-NP yield during five subsequent runs of dephosphorylation reaction (8 h for each run). It was found that the yield of p-NP had a significant drop for the nanooctahedra and nanorods, whereas the loss of catalytic activity for the nanospheres was almost negligible. At the end of the recyclability test, approximately 1%, 7% and 10% drop in yield of p-NP was observed for the nanospheres, nanooctahedra and nanorods, respectively, as compared to 7% for the commercial CeO₂. The yield of p-NP from the CeO₂ nanocubes was consistently low and never exceeded ˜5% throughout the recyclability studies.

From TEM images collected for the CeO₂ nanocrystals after the tests, it can be seen that the difference in recyclability is well correlated to the extents of particle aggregation (FIG. 6(b-f)). The nanorods and nanocubes had the most severe aggregation, followed by the nanooctahedra and commercial CeO₂, whereas the nanospheres exhibited negligible aggregation. These results are in line with the consensus of size- and shape-dependent colloidal assembly and stability of nanocrystals.

It will be recognized from the foregoing that the present disclosure describes systematic analysis of catalytic dephosphorylation using CeO₂ nanocrystals. CeO₂ nanocrystals of different shapes were synthesized and demonstrated as “artificial phosphatases” to cleave the phosphate ester bond inpara-nitrophenyl phosphate (p-NPP) and release free phosphate anions. The dephosphorylation reaction kinetics, including rate constant and activation energy, as well as the catalyst recyclability, were found to be dependent on the particle shape and surface defects, with the catalytic activity following the trend nanosphere>nanooctahedra>nanorod>nanocube. By correlating the reaction kinetics to the surface structure analysis based on O₂-TPD, the active sites for dephosphorylation were demonstrated to be associated with oxygen vacancies on the surface of the CeO₂ nanocrystals. Thus, the disclosure illustrates utility of catalytic dephosphorylation for recovery of phosphorus nutrients from biomass and organic wastes.

The following provides a description of the materials and methods used to produce the results presented herein.

Materials and Chemicals. The following chemicals were purchased and used as-received without further purification: cerium(IV) oxide (nanopowder, ≥99.5%, Alfa Aesar), cerium(III) nitrate hexahydrate (Ce(NO₃)₃-6H₂O, 99% trace metals basis, Aldrich), hexamethylenetetramine ((CH₂)₆N₄, HMT, ≥99%, Aldrich), hydrogen peroxide (H₂O₂, ACS grade, 30 wt %, Fisher), sodium hydroxide (NaOH, 99.1%, Fisher), para-nitrophenol (C₆H₅NO₃, p-NP, ≥99%, Aldrich), para-nitrophenyl phosphate disodium salt hexahydrate (C₆H₄NO₆PNa₂-6H₂O, p-NPP, ≥99%, Sigma), L-ascorbic acid (C₆H₈O₆, reagent grade, Sigma), ammonium molybdate tetrahydrate ((NH₄)₆Mo₇O₂₄-4H₂O, ACS reagent, 81.0-83.0% MoO₃ basis, Sigma-Aldrich), sodium phosphate dibasic (Na₂HPO₄, ≥98.5%, Sigma), sulfuric acid (H₂SO₄, ACS grade, BDH), and anhydrous ethanol (C₂H₅OH, 200 proof, ACS/USP grade, Pharmco-Aaper). Deionized water was collected from an ELGA PURELAB flex apparatus.

Synthesis of CeO₂ Nanocrystals. For the synthesis of CeO₂ nanospheres, 1 mmol of cerium nitrate hexahydrate (Ce(NO₃)₃.6H₂O) and 32 mL of 0.078 M NaOH were added to a 100 mL reaction flask. The mixture was stirred at 700 rpm for 22 h at 25° C. in air. The CeO₂ nanospheres were collected and washed with ethanol and deionized water three times by centrifugation at 10,000 g for 10 min and were dispersed in deionized water for further use.

For the synthesis of CeO₂ nanooctahedra, a 100 mL reaction flask was charged with 5 mL of 0.0375 M Ce(NO₃)₃, 1 mL of 0.5 M hexamethylenetetramine (HMT), and an additional 5 mL of deionized water. The mixture was stirred at 700 rpm in air and heated to 75° C. for 3 h. The solution slowly turned from clear to turbid white, indicating the formation of CeO₂ nanocrystals. The precipitated CeO₂ nanooctahedra were annealed in static air at 200° C. for 12 h to remove the HMT surfactant.

Similar to the synthesis of nanospheres, CeO₂ nanorods were synthesized by mixing 5 mL of 0.4 M Ce(NO₃)₃ and 35 mL of 9 M NaOH in a Teflon-lined stainless steel autoclave, and then heating this mixture at 100° C. for 24 h. CeO₂ nanocubes were made by a similar approach to the nanorods with 5 mL of 1.5 M Ce(NO₃)₃ and 35 mL of 6 M NaOH.

Material Characterization. Transmission electron microscopy (TEM) images were taken on an FEI Tecnai 12 operating at 100 kV. High-resolution transmission electron microscopy (HRTEM) images were captured on a Phillips CM 300 FEG operating at 300 kV. TEM samples were prepared by dispersing the CeO₂ nanocrystals in ethanol and depositing droplets of the obtained solution on Cu grids (400 mesh, coated with carbon film), with the solvent evaporated under ambient conditions. X-ray diffraction (XRD) patterns were obtained from a PANalytical X′Pert³ X-ray diffractometer equipped with a Cu Kα radiation source (λ=1.5406 Å). X-ray photoelectron spectroscopy (XPS) measurements were taken on a PHI 5400 X-ray photoelectron spectrometer equipped with an Al Kα X-ray source. Nitrogen adsorption measurements were measured on dried powders of CeO₂ nanocrystals using a Micromeritics ASAP 2020, with the nanocrystals degassed under vacuum for 8 h at 180° C. and measured at a temperature ramping rate of 5° C/min. Specific surface areas (SSA) were calculated according to the Brunauer-Emmett-Teller (BET) theory.

Catalytic Studies. A stock solution of p-NPP was first prepared at a concentration of 0.2 mg/mL. An aliquot (10 mL) of this stock solution was mixed with 3.5 mg of CeO₂ nanocrystals which was then heated to the desired reaction temperature (ranging from 3 to 95° C.) using an ice bath or a hot plate. The reaction was carried out under ambient conditions. As the reaction proceeded, the solutions turned from turbid white to turbid yellow, indicating the formation of para-nitrophenol (p-NP). At different time intervals, 0.5 mL of the reaction solution was collected, to which 0.5 mL of ethanol was added. After removing the CeO₂ catalyst by centrifugation (16,000 rpm for 5 min), the collected solution was further treated with NaOH (1%) %) to adjust the pH and convert para-nitrophenol to para-nitrophenolate. Ultraviolet-visible absorption spectra were collected by using a SpectraMax Plus 384 spectrometer to analyze the concentrations of p-NPP and p-NP.

The concentration of phosphate produced from the dephosphorylation reaction was characterized by using a modified molybdenum blue assay (see more details in the Supporting Information). A stock solution of 0.088 mg/mL of sodium phosphate was first prepared, and then diluted in a series (0.5 dilution factor) to create the standards for calibration. 200 μL of the assay was used for 1 mL of reaction solution or phosphate standard. UV-Vis absorption was measured at 890 nm and the absorbance was used to analyze the concentration of phosphorus.

In performing the recyclability studies, the same catalyst loading and reaction concentration was maintained. After each run of dephosphorylation reaction for 8 h, the catalyst was isolated from the reaction mixture by centrifugation and re-dispersed in deionized water by sonication, which was then added to a fresh p-NPP solution for the next run.

Temperature-Programmed Desorption of Oxygen (O₂-TPD). O₂-TPD patterns were collected on CeO₂ nanocrystal powders (ca. 100 mg) using a gas chromatograph (GC-2010 Plus equipped with a Barrier Ionization Discharge (BID) detector, Shimadzu). Dried catalysts were loaded in a plug flow reactor with a quartz tube of ⅛″ in diameter, and pretreated in He at 300° C. for 1 h. After cooled down to room temperature, oxygen adsorption was performed by flowing O₂ (20 mL/min) for 30 min. The physically adsorbed oxygen was removed by purging with He for ˜60 min. For desorption, the temperature was increased from room temperature to 600° C. at a ramping rate of 5° C/min. The desorbed oxygen was carried out by a flow of He (20 mL/min) and analyzed by the GC-BID.

S1. Molybdenum Blue Assay. A 5.0 N sulfuric acid solution was prepared by diluting 7 mL of concentrated H2504 to 50 mL with deionized water. A 4.0 wt % ammonium molybdate solution was prepared by dissolving 400 mg of (NH₄)₆Mₒ₇O₂₄-4H₂O in 10 mL of deionized water. A 0.1 M L-ascorbic acid solution was prepared by dissolving 176 mg of C₆H₈O₆ in 10 mL of deionized water.

5 mL of the 4.0 wt % ammonium molybdate solution was added to 17 mL of the 5.0 N sulfuric acid solution and gently stirred. The solution remained clear. Then 10 mL of the 0.1 M L-ascorbic acid was added and the solution turned golden yellow as it was gently mixed. The molybdenum blue assay was used immediately after preparation. It was found to lose its efficacy when it was left overnight.

Stock phosphate solutions were prepared by dissolving 4.4 mg of Na₂HPO₄ in 50 mL of deionized water. A series of dilutions (0.5 dilution factor) were carried out to prepare the phosphate standards. To 1 mL of each standard, 200 μL of the reagent mixture was added. The standards slowly turned blue. 200 μL of each standard was dispensed to a microplate for ultraviolet-visible (UV-Vis) spectroscopy analysis at 890 nm and a calibration curve for phosphate concentration was constructed. To each 1 mL supernatant extracted during the model dephosphorylation reactions, 200 μL of the reagent mixture was added. The supernatants quickly changed color from yellow to clear to blue and were analyzed via UV-Vis at 890 nm to quantify the amount of phosphate present.

S2. Kinetic Calculations. Molar quantities (n, in mmol) of p-NPP and p-NP were calculated by using UV-Vis calibration equations (Figure S6). Yield of p-NP (in %) was determined according to the following equation:

$$Yield of p - NP = \frac{{n}_{t , p - NP}}{{n}_{0 , p - NPP}} \times 100 \qquad ( S 1 )$$

where: n_{t, p-NP}=amount of p-NP (in mmol) at reaction time, t (in hours (h)).

n_{0, p-NPP}=initial amount of p-NPP, or the theoretical yield of p-NP (in mmol).

The initial molar amount of p-NPP also corresponded to the maximum theoretical amount of p-NP that could be produced.

First order rate constants, k (in h⁻¹), were calculated by monitoring the conversion of p-NPP top-NP. A linear plot using the following equation was used to determine k:

$$ln ( \frac{{n}_{0 , p - NPP}}{{n}_{t , p - NPP}} ) = kt \qquad ( S 2 )$$

where: n_{t, p-NPP} =amount of p-NPP (in mmol) at reaction time, t (in h)

t=reaction time (in h).

To make comparisons among the various morphologies, k was normalized by catalyst loading (k^{m,} in g⁻¹-h⁻¹) and by surface area (k^{s,} in m⁻²-h⁻¹) according to the following equations:

$$\begin{matrix}{k}_{m} = \frac{k}{{m}_{cat}} & ( S 3 ) \\ {k}_{s} = \frac{k}{{m}_{cat} {SA}_{cat}} & ( S 4 )\end{matrix}$$

where: m_{cat}=mass loading of CeO₂ nanocatalyst (in grams (g))

SA_{cat}=surface area of CeO₂ nanocatalyst (in m²/g)

Activation energy, Eₐ(in kJ/mol), was calculated according to the linearized

Arrhenius equation:

$$ln k = - \frac{{E}_{a}}{RT} + ln A \qquad ( S 5 )$$

where: T=reaction temperature (in K)

R=universal gas constant (8.314 J mol⁻¹ K⁻¹)

A=pre-exponential factor.

S3. Temperature-Programmed Desorption of Oxygen (O₂-TPD). O₂-TPD measurements were used to calculate the density of surface oxygen vacancy sites for each CeO₂ nanocatalyst. The adsorbed molecular oxygen peak (ad-O₂) directly corresponded to the molar quantity of O₂ adsorbed in the oxygen vacancies. These peaks were integrated and peak areas were compared to a calibration curve constructed from O₂/He streams of known concentrations. This allowed for a direct quantification of moles of desorbed O₂ from the vacancies which directly equaled the moles of surface vacancy sites in each sample, ⁿ_{vac}. An amount of 100 mg, ^{m}_{TPD}, was used for each CeO₂ sample. Surface vacancy density, ρ_{vac} (in sites/m²), was calculated according to:

$${ρ}_{vac} = \frac{{n}_{vac} {N}_{A}}{{m}_{TPD} {SA}_{cat}} \qquad ( S 6 )$$

where: N_{A}=Avogadro's number.

The turnover frequency (TOF, in h⁻¹) for each nanocatalyst at 25° C. was calculated by taking the ratio of the molecular reaction rate over the density of surface oxygen vacancies:

$$TOF = \frac{{kn}_{f , p - NP} {N}_{A}}{{ρ}_{vac}} \qquad ( S 7 )$$

where: n_{f, p-NP}=amount of p-NP produced after reaction.

### EXAMPLE 2

This example provides a description of dephosphorylation catalyzed by ceria nanoparticles (e.g., nanocrystals).

Disclosed herein is the effect of pH on the catalytic dephosphorylation over CeO₂ nanocrystals. Ce₂ nanospheres and nano-octahedra were applied to dephosphorylatepara-nitrophenyl phosphate (p-NPP) in aqueous solutions. The solution pH was tuned from 1 to 11 by using phosphate buffers to examine the dependence of catalytic activity on the pH. The established trend was further interpreted via the availability of free oxygen vacancies on the surface of the CeO₂ nanocrystals, with the latter determined by combining temperature-programmed desorption of oxygen (O₂-TPD) and phosphate adsorption measurements. Our results suggest that the desorption of phosphate is likely the rate-determining step in the catalytic dephosphorylation on CeO₂ nanocrystals, on which the oxygen vacancies serve as the active sites.

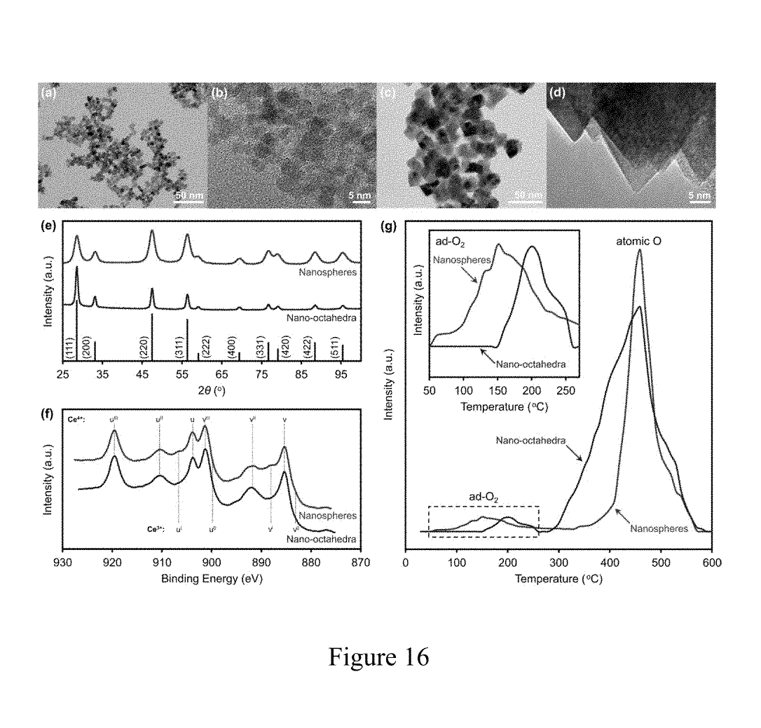

Synthesis and Characterization of CeO₂ Nanocrystals. CeO₀₂ nanocrystals were prepared via hydrothermal reactions of Ce(NO₃)₃-6H₂O with NaOH. FIGS. 16a-d show transmission electron microscopy (TEM) and high-resolution TEM (HRTEM) images of the CeO₂ nanocrystals employed in this study. The nano-octahedra have an average size of 18.0±1.6 nm with (111) facets preferentially exposed on the surface. The nanospheres have an average diameter of 4.0±0.6 nm, but without preferential exposure of certain facets on the surface. The X-ray diffraction (XRD) patterns collected for the CeO₂ nanocrystals exhibit major peaks at 28.5°, 33.1°, 47.5° and 56.3° , corresponding to the (111), (200), (220) and (311) planes, respectively, of the fluorite structure of ceria (space group Fm3̅m, JCDPS No. 65-2795) (FIG. 16e). X-ray photoelectron spectroscopy (XPS) analysis at the Ce 3d edge reveals the presence of Ce³⁺ in the nanocrystals, which are likely associated with oxygen vacancies in the nanocrystals (FIG. 16f). It is shown by temperature-programmed desorption of oxygen (O₂-TPD) that oxygen vacancies are exposed on the surface of the CeO₂ nanocrystals, and the number density is determined to be 2.6×10⁶ and 4.3×10⁶ sites/m² for the nano-octahedra and nanospheres, respectively. These defects are believed to be the active sites for the catalytic dephosphorylation reaction.

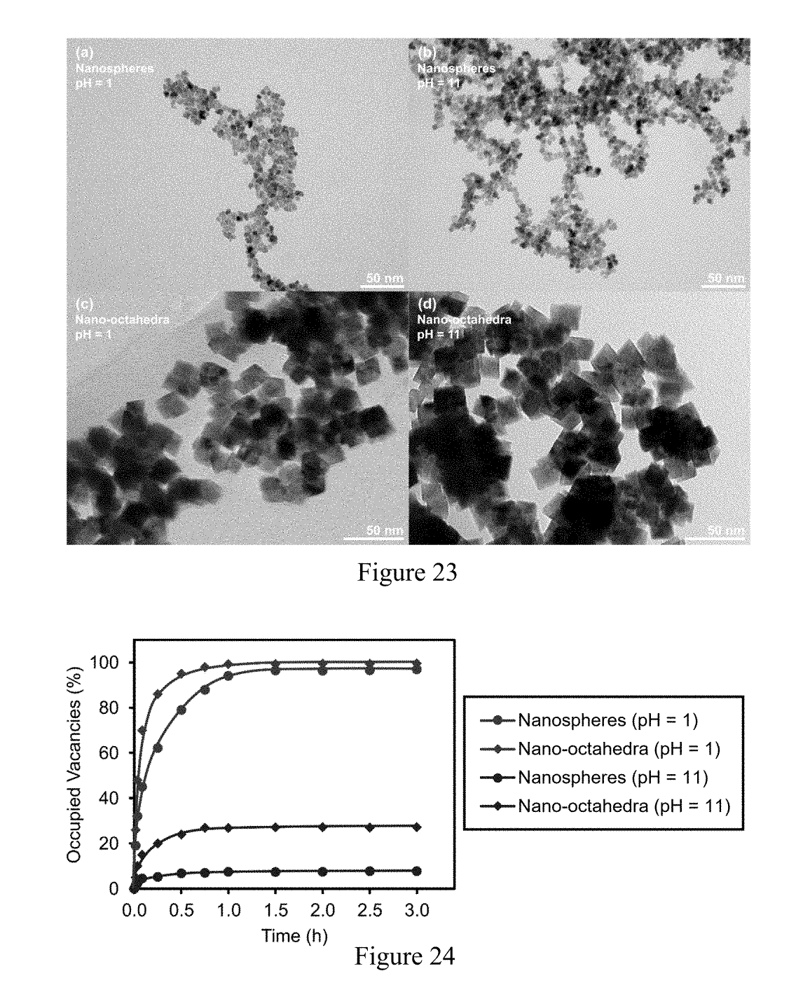

pH-dependent Dephosphorylation Activities. The CeO₂ nanocrystals were applied as catalysts for the dephosphorylation of para-nitrophenyl phosphate (p-NPP) in aqueous solutions in the temperature range of 5-95° C. Due to its chromogenic properties, p-NPP and its hydrolysis productpara-nitrophenol (p-NP, ionized to para-nitrophenolate after a pH adjustment) exhibit distinct optical absorption at 310 nm and 400 nm, respectively, allowing for characterization of reaction products using UV-Vis absorption spectroscopy. Meanwhile, the phosphate groups released into the solution are analyzed by using a molybdenum blue assay. To examine the effects of pH on dephosphorylation, aliquots of HNO₃ or NaOH solutions are added to the solutions of p-NPP (0.2 mg/mL) to adjust the pH from 1 to 11 (FIG. 21). It is confirmed by TEM imaging that the catalysts preserved their morphologies after the catalytic studies throughout the pH range (FIG. 23).

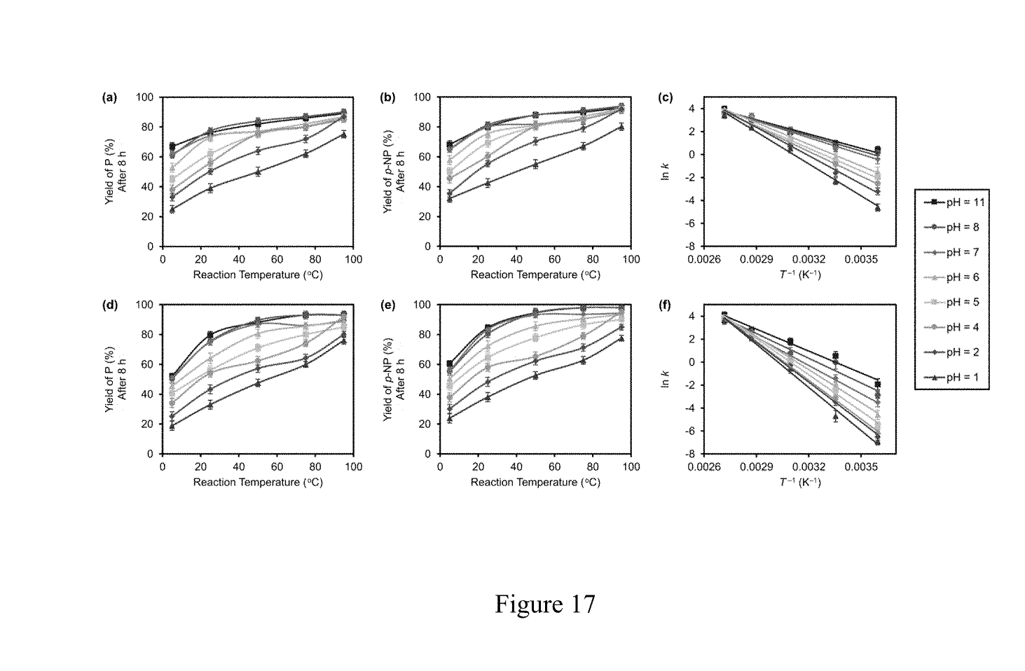

FIGS. 17 summarizes the results of the catalytic studies for the dephosphorylation reaction at different pH and room temperature (25° C.). Across all the studied pH values, both the yields of P andp-NP increase with reaction temperature, indicating a positive activation energy for the hydrolysis reaction (FIGS. 17 a-d). The dephosphorylation of p-NPP yeilds nearly equivalent amounts of P andp-NP, suggesting that no undesired side reactions take place and the products are completely desorbed from the catalyst surface. An interesting observation is the higher yields of P and p-NP at higher pH. At the room temperature (25° C.), the yield of P is 25.0±2.5% and 33.0±2.1%, 38.1±2.0%, 45.2±2.8%, 52.8±2.2%, 62.0±2.1%, 61.2±2.0% and 67.0±2.1% for pH values of 1, 2, 4, 5, 6, 7, 8 and 11, respectively. The differences due to pH are more significant at lower temperatures. At 95° C., however, yields of P all reach ˜85-90% (with the only exception being ˜75% for a pH of 1). Similar trends are observed for the CeO₂ nano-octahedra. At 5° C., yields of P over CeO₂ nano-octahedra are 18.8±2.9%, 25.2±3.0%, 34.0±2.8%, 40.5±2.6%, 45.4±3.0%, 50.4±2.3%, 51.0±2.8%, 52.0±2.5% for pH values of 1, 2, 4, 5, 6, 7, 8 and 11, respectively. At each temperature, first-order rate constants, k, are calculated based on the changes in concentration of p-NPP and Arrhenius plots are constructed. As shown in FIGS. 17c and 17f for the nanospheres and nano-octahedra, respectively, the slope of these plots decrease as alkalinity increases. This corresponds to reaction activation energy, which is discussed below.

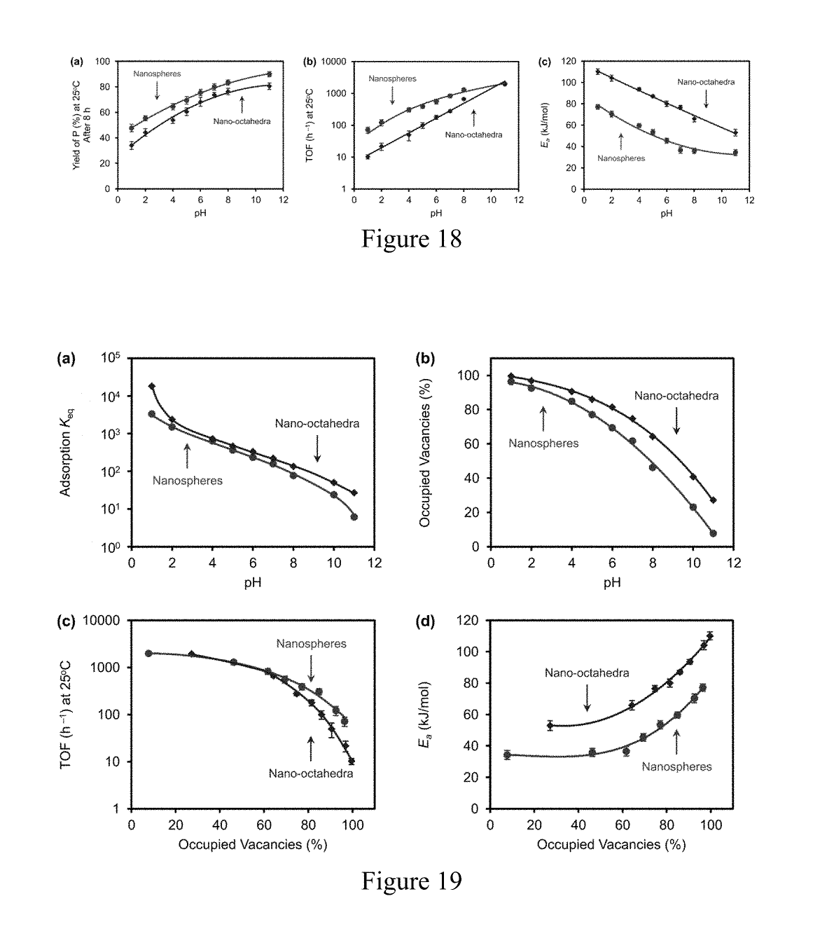

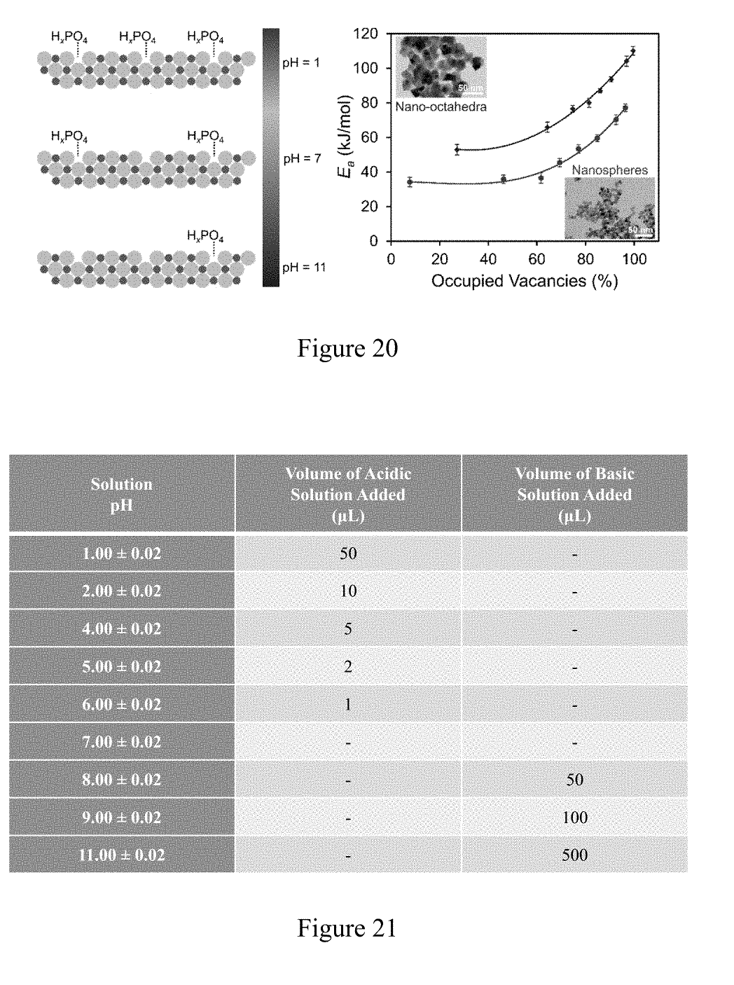

FIG. 18a illustrates the changes in yield of P at 25° C. after 8 h of catalytic treatment as a function of pH. For the CeO₂ nanospheres, yields of P at 25° C. are 39.0±2.2%, 50.3±3.0%, 56.0±2.4%, 62.0±2.3%, 73.0±3.0%, 74.0±2.8%, 77.4±2.4%, 87.4±2.2% for pH values of 1, 2, 4, 5, 6, 7, 8 and 11, respectively. For the CeO₂ nano-octahedra, a similar trend is observed with yields of P generally about ˜5% lower than the nanospheres with 34.0±2.9%, 44.2±3.1%, 53.9±2.5%, 60.5±2.8%, 68.2±3.5%, 73.6±2.1%, 76.4±2.5%, 80.5±2.6% for pH values of 1, 2, 4, 5, 6, 7, 8 and 11, respectively. FIG. 18b illustrates the dependency of activation energy, Eₐ, and on reaction solution pH. Activation energy values for CeO₂ nanospheres derived from the aforementioned Arrhenius plots are 77.1±2.2, 70.3±2.8, 59.6±1.9, 53.4±2.5, 45.5±2.4, 36.6±3.0, 35.9±2.5 and 34.2±2.8 kJ/mol for pH values of 1, 2, 4, 5, 6, 7, 8 and 11, respectively. Again, a similar trend is found for the CeO₂ nano-octahedra, with higher activation energies of 110.1±2.5, 104.1±2.9, 93.6±1.5, 87.0±1,2, 80.0±2.6, 76.6±1.9, 65.9±2.9 and 52.9±3.1 kJ/mol for pH values of 1, 2, 4, 5, 6, 7, 8 and 11, respectively. FIG. 18c shows turnover frequency (TOF) at 25° C., which is calculated by normalizing molecular reaction rates by surface oxygen vacancy density (identified as the active site for dephosphorylation in a previous report). For the CeO₂ nanospheres TOF is 71±16 h⁻¹, 121±26 h⁻¹, 307±46 h⁻¹, 392±60 h⁻¹, 556±90 h⁻¹, 827±111 h⁻¹, 1284±147 h⁻¹ and 1983±155 h⁻¹ for pH values of 1, 2, 4, 5, 6, 7, 8 and 11, respectively. Yet again, a similar trend is observed for the CeO² nano-octahedra, TOF is 10±2 h⁻¹, 22±6 h⁻¹, 49±17 h⁻¹, 99±20 h−1, 178±26 h⁻¹, 277±23 h⁻¹, 660±51 h⁻¹ and 1931±61 h⁻¹ for reaction solutions with pH values of 1, 2, 4, 5, 6, 7, 8 and 11, respectively. Thus, solution pH plays a significant role in the kinetics of aqueous dephosphorylation.

Phosphate Adsorption on CeO₂ Nanocrystals. To study these effects further, adsorption of phosphate on CeO₂ is examined. CeO₂ nanocrystals are added to phosphate solutions buffered to pH values in the range of 1-11. Equilibrium adsorption of phosphate is found to occur at ˜1.5 h (FIG. 24) and all phosphate solutions are stirred for 3 h to ensure equilibrium conditions are reached. Phosphate concentrations are monitored with a molybdenum blue assay and solution pH is recorded before and after adsorption. See the Methods for further details.

FIG. 19a shows the adsorption equilibrium constants, K_{eq}, for each solution pH. Several reasonable assumptions are made: first, that the phosphate adsorption occurs via the coordination of phosphoryl oxygen in surface oxygen vacancies (denoted as □ below); second, that one phosphate group can occupy one vacancy at a time; third, that adsorption of all forms of phosphate is possible, including H₃PO₄, H₂PO₄⁻, HPO₄²⁻ and PO₄³⁻(collectively denoted as HₓPO₄); and fourth, that the equilibrium reaction is:

HₓPO₄+□⇄HₓPO₄ . . . □ (1)

Adsorption equilibrium constants were found to decrease as a function of increasing alkalinity. A sharper decrease was observed in the acidic pH range, with K_{eq} found to drop from 3331.7 to 1501.0 to 639.2 at pH values of 1, 2 and 4, respectively for the CeO₂ nanospheres. In the alkaline regime, the decrease in adsorption equilibrium constant is not as drastic, with K_{eq} observed to be 77.1, 23.8, and 6.1 for pH values of 8, 10, and 11, respectively. Combined with the assumptions above, these adsorption equilibrium constants correlate to the number of occupied surface oxygen vacancies and is plotted in FIG. 19b. 96.4%, 92.5%, 84.8%, 77.1%, 69.4%, 61.7%, 46.3%, 23.1% and 7.7% of the vacancies on CeO₂ nanospheres are occupied for solutions with pH values of 1, 2, 4, 5, 6, 7, 8, 10 and 11, respectively. Similarly, 99.6%, 96.9%, 90.5%, 86.0%, 81.5%, 74.7%, 64.3%, 40.7% and 27.2% of the vacancies on CeO₂ nano-octahedra are occupied for solutions with pH values of 1, 2, 4, 5, 6, 7, 8, 10 and 11, respectively.

FIG. 19c-d demonstrates the relationship between occupied vacancies and kinetic activity (the aforementioned values of activation energy and turnover frequency). It is evident that vacancy occupation reduces catalytic activity. For instance, when 7.7% of vacancies on the nanospheres are occupied by phosphate, the activation energy and TOF are observed to be 34.2±2.8 kJ/mol and 1983±155 h⁻¹, respectively. When 96.4% of vacancies are occupied on the nanospheres by phosphate, the activation energy and TOF are observed to be 77.1±2.2 kJ/mol and 71±16 h⁻¹, respectively. The same trends are evident for CeO₂ nano-octahedra.

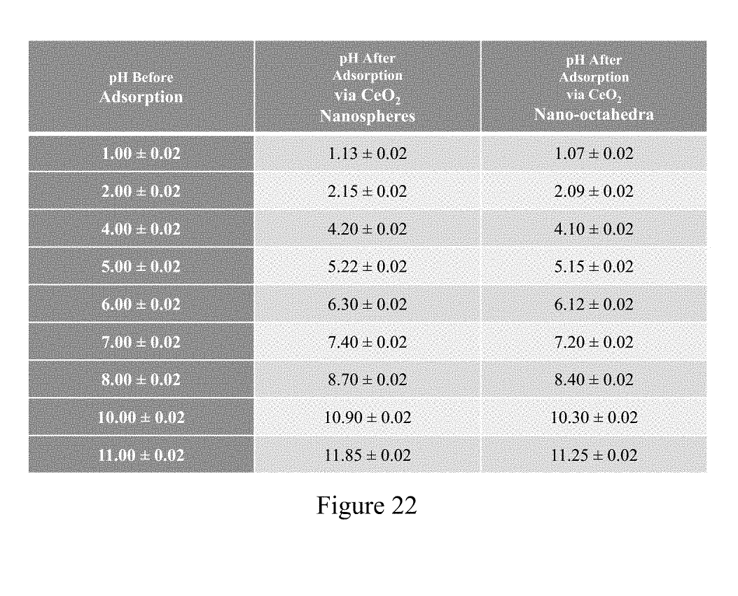

Previously, it has been shown that adsorption of phosphate on CeO₂ (and other metal oxides) is highly pH-dependent. Phosphate has been shown to bind more strongly to CeO₂ at acidic pH values. It was hypothesized that lower solution pH reduces the coordination of CeO₂ with surface hydroxyls, allowing for a stronger ionic exchange process with phosphate anions. To verify this phenomenon, the change in solution pH before and after adsorption on CeO₂ is measured (FIG. 22). The consistent increase in pH observed during this process indicates that a ligand exchange phenomenon is likely. It suggests that at lower pH, hydroxide is less prevalent on the surface of CeO₂ and that adsorption of phosphate occurs more readily.

From these results, it is proposed that the persistent adsorption of phosphate to the surface of CeO₂ limits the kinetics of dephosphorylation. Increased adsorption of phosphate to

CeO₂ poisons the catalyst and thus hinders the adsorption of new reactants, reducing the observed TOF. The observance of increasing activation energy coupled with increased phosphate poisoning further indicates that product desorption is the rate-determining step in aqueous dephosphorylation. Adsorption is more prevalent at lower pH, thus explaining the reduced kinetic activity observed in acidic reaction solutions.

Furthermore, there is a difference in adsorption between the nanospheres and nano-octahedra regarding the stability of interactions between adsorbates and oxygen vacancies. FIG. 24 shows that adsorption of phosphate on CeO₂ nano-octahedra proceeds faster than on the nanospheres. Furthermore, O₂-TPD results in FIG. 16g show that the desorption of adsorbed molecular oxygen within the surface oxygen vacancies begins at a lower temperature for the nanospheres (˜50° C.) than for the nano-octahedra (˜150° C.). This evidence indicates: (1) that the stability and the strength of interactions between adsorbates and oxygen vacancies follows the trend of nano-octahedra>nanospheres; (2) that such trends in stability are likely due to steric hindrance effects caused by stepping and truncated facets on the nanospheres that are not as prevalent on the nano-octahedra; and (3) that higher-performing CeO₂ nanocatalysts for dephosphorylation have weaker adsorbate-vacancy interactions resulting in less surface poisoning and greater reaction turnover.

Conclusion. Catalytic dephosphorylation of p-NPP was studied over CeO₂ nanospheres and nano-octahedra at various solution pH values. Results indicate that increasing reaction solution alkalinity improves catalytic performance of aqueous dephosphorylation. The effects of reaction solution pH are attributed to increasing phosphate adsorption to the surface of CeO₂ at increasing acidic pH values. Combining the studies of catalytic tested with phosphate adsorption at various pH values, it is considered that product desorption is the rate-determining step in aqueous dephosphorylation over CeO₂ nanocrystals.

Methods. Materials and Chemicals. The following chemicals were purchased and used as-received without further purification: cerium(III) nitrate hexahydrate (Ce(NO₃)₃-6H₂O, 99% trace metals basis, Aldrich), hexamethylenetetramine ((CH₂)₆N₄, HMT, ≥99%, Aldrich), sodium hydroxide (NaOH, 99.1%, Fisher), para-nitrophenol (C₆H₅NO₃, p-NP, ≥99%, Aldrich), para-nitrophenyl phosphate disodium salt hexahydrate (C₆H₄NO₆PNa₂-6H₂O, p-NPP, ≥99%, Sigma), L-ascorbic acid (C₆H₈O₆, reagent grade, Sigma), ammonium molybdate tetrahydrate ((NH₄)₆Mo₇O₂₄-4H₂O, ACS reagent, 81.0-83.0% MoO₃ basis, Sigma-Aldrich), nitric acid (HNO₃, certified ACS plus, 68.0-70.0%, Fisher), sodium phosphate dibasic (Na₂HPO₄, ≥98.5%, Sigma), sulfuric acid (H₂SO₄, ACS grade, BDH), and anhydrous ethanol (C₂H₅OH, 200 proof, ACS/USP grade, Pharmco-Aaper). Deionized water was collected from an ELGA PURELAB flex apparatus.

Synthesis of CeO₂ Nanocrystals. To synthesize CeO₂ nanospheres, 1 mmol of cerium nitrate hexahydrate (Ce(NO₃)₃-6H₂O) and 32 mL of 0.078 M NaOH is added to a 100 mL reaction flask. The mixture is stirred at 700 rpm for 22 h at 25° C. in static air. To synthesize CeO₂ nano-octahedra, a 100 mL reaction flask is charged with 5 mL of 0.0375 M Ce(NO₃)₃, 1 mL of 0.5 M hexamethylenetetramine (HMT) and an additional 5 mL of deionized water. The mixture is stirred at 700 rpm in air and heated to 75° C. for 3 h. The CeO₂ nanocrystals are collected and washed with ethanol and deionized water three times by centrifugation at 10,000g for 10 min each, calcined at 200° C. for 12 h and dispersed in deionized water for catalytic testing.

Material Characterization. Transmission electron microscopy (TEM) images were taken on an FEI Tecnai 12 operating at 100 kV. High-resolution TEM (HRTEM) images were captured on a Phillips CM 300 FEG operating at 300 kV. TEM samples were prepared by dispersing the CeO₂ nanocrystals in ethanol and depositing droplets of the obtained solution on Cu grids (400 mesh, coated with carbon film), with the solvent evaporated under ambient conditions. XRD patterns were obtained from a PANalytical X′Pert3 X-ray diffractometer equipped with a Cu Kα radiation source (λ=1.5406 Å). XPS measurements were taken on a PHI 5400 X-ray photoelectron spectrometer equipped with an Al Kα X-ray source. Nitrogen adsorption measurements were measured on dried powders of CeO₂ nanocrystals using a Micromeritics ASAP 2020 with the nanocrystals degassed under vacuum for 8 h at 180° C. and measured at a temperature ramping rate of 5° C/min. Specific surface area (SSA) was calculated according to the BET theory. O₂-TPD patterns were collected on CeO₂ nanocrystal powders (˜100 mg) using a gas chromatograph (GC-2010 Plus equipped with a barrier ionization discharge (BID) detector, Shimadzu). Dried catalysts were loaded in a plug flow reactor with a quartz tube ⅛″ in diameter and pretreated in He at 300° C. for 1 h. After being cooled to room temperature, oxygen adsorption was performed by flowing O₂ (20 mL/min) for 30 min. The physically adsorbed oxygen was removed by purging with He for ˜60 min. For desorption, the temperature was increased from room temperature to 600° C. at a ramping rate of 5° C/min. The desorbed oxygen was carried out by a flow of He (20 mL/min) and analyzed by the GC-BID.

Catalytic Studies. 10 mL of a 0.2 mg/mL solution of p-NPP was adjusted to the desired pH (see FIG. 21) which was then heated to the desired reaction temperature (5-95° C.) using an ice bath or a hot plate. 3.5 mg of CeO₂ nanocrystals was added, initiating the reaction.

As the reaction proceeded, the solutions turned from turbid white to turbid yellow, indicating the formation of para-nitrophenol (p-NP). At different time intervals, 0.5 mL of the reaction solution was collected, to which 0.5 mL of ethanol was added as a precipitating solution. After removing the CeO₂ catalyst by centrifugation (16,000 rpm for 5 min), the collected solution was further treated with NaOH (1%) to adjust the pH back to the basic range and convert para-nitrophenol to para-nitrophenolate. Ultraviolet-visible (UV-vis) absorption spectra were collected by using a SpectraMax Plus 384 spectrometer to analyze the concentrations of p-NPP and p-NP at 311 and 400 nm, respectively. The concentration of phosphate was characterized by using a modified molybdenum blue assay. UV-vis absorption was measured at 890 nm and the absorbance was used to analyze the concentration of phosphorus.

Phosphate Adsorption Studies. 10 mL of a 0.2 mg/mL solution of Na₂HPO₄ was adjusted to the desired pH (see FIG. 21). 3.5 mg of CeO₂ nanocrystals was added, initiating the adsorption process. Samples were extracted at specified time intervals according to the same procedure for kinetic tests. The same molybdenum blue assay was used to determine the concentration of phosphorus.

S1. Molybdenum Blue Assay. A 5.0 N sulfuric acid solution was prepared by diluting 7 mL of concentrated H2504 to 50 mL with deionized water. A 4.0 wt % ammonium molybdate solution was prepared by dissolving 400 mg of (NH₄)₆Mo₇O₂₄-4H₂₀ in 10 mL of deionized water. A 0.1 M L-ascorbic acid solution was prepared by dissolving 176 mg of C₆H₈O₆ in 10 mL of deionized water.

5 mL of the 4.0 wt % ammonium molybdate solution was added to 17 mL of the 5.0 N sulfuric acid solution and gently stirred. The solution remained clear. Then 10 mL of the 0.1 M L-ascorbic acid was added and the solution turned golden yellow as it was gently mixed. The molybdenum blue assay was used immediately after preparation. It was found to lose its efficacy when it was left overnight.

Stock phosphate solutions were prepared by dissolving 4.4 mg of Na₂HPO₄ in 50 mL of deionized water. A series of dilutions (0.5 dilution factor) were carried out to prepare the phosphate standards. To 1 mL of each standard, 200 μL of the reagent mixture was added. The standards slowly turned blue. 200 μL of each standard was dispensed to a microplate for ultraviolet-visible (UV-Vis) spectroscopy analysis at 890 nm and a calibration curve for phosphate concentration was constructed. To each 1 mL supernatant extracted during the model dephosphorylation reactions, 200 μL of the reagent mixture was added. The supernatants quickly changed color from yellow to clear to blue and were analyzed via UV-Vis at 890 nm to quantify the amount of phosphate present.

Molar quantities (n, in mmol) of p-NPP and p-NP were calculated by using UV-Vis calibration equations. Yield of p-NP (in %) was determined according to the following equation:

$$Yield of p - NP = \frac{{n}_{t , p - NP}}{{n}_{0 , p - NPP}} \times 100 \% \qquad ( S 1 )$$

Where: n_{t, p-NP}=amount of p-NP (in mmol) at reaction time, t (in h)

n_{0, p-NPP} =initial amount of p-NPP, or the theoretical yield of p-NP (in mmol)

The initial molar amount of p-NPP also corresponded to the maximum theoretical amount of p-NP that could be produced.

First order rate constants, k (in h⁻¹), were calculated by monitoring the conversion of p-NPP top-NP. A linear plot using the following equation was used to determine k:

$$ln ( \frac{{N}_{0 , p - NPP}}{{n}_{t , p - NPP}} ) = kt \qquad ( S 2 )$$

Where: n_{t, p-Npp}=amount of p-NPP (in mmol) at reaction time, t (in h)

t=reaction time (in h)

To make comparisons among the various morphologies, k was normalized by catalyst loading (kₘ, in g⁻¹-h^{-h}) and by surface area (kₛ, in m⁻²-h⁻¹) according to the following equations:

$$\begin{matrix}{k}_{m} = \frac{k}{{m}_{cat}} & ( S 3 ) \\ {k}_{s} = \frac{k}{{m}_{cat} {SA}_{cat}} & ( S4 )\end{matrix}$$

Where: m_{cat}=mass loading of CeO₂ nanocatalyst (in g)

SA_{cat}=surface area of CeO₂ nanocatalyst (in m²/g)

Activation energy, Eₐ (in kJ/mol), was calculated according to the linearized Arrhenius equation:

$$ln k = - \frac{{E}_{a}}{RT} + ln A \qquad ( S 5 )$$

Where: T=reaction temperature (in K)

R=universal gas constant (8.314 J/mol-K)

A=pre-exponential factor

Oxygen temperature-programmed desorption (O₂-TPD) measurements were used to calculated the density of surface oxygen vacancy sites for each CeO₂ nanocatalyst. The adsorbed molecular oxygen peak (ad-O₂) directly corresponded to the molar quantity of O₂ adsorbed in the oxygen vacancies. These peaks were integrated and peak areas were compared to a calibration curve constructed from O₂/He streams of known concentrations. This allowed for a direct quantification of moles of desorbed O₂ from the vacancies which directly equaled the moles of surface vacancy sites in each sample, n_{vac}. About 100 mg, m_{TPD}, was used for each CeO₂ sample. Surface vacancy density, ρ_{vac} (in sites/m²), was calculated according to:

$${ρ}_{vac} = \frac{{n}_{v a c} {N}_{A }}{{m}_{TPD} {SA}_{cat}} \qquad ( S 6 )$$

Where: N_{A}=Avogadro' s number

The turnover frequency (TOF, in h⁻¹) for each nanocatalyst at 25° C. was calculated by taking the ratio of the molecular reaction rate over the density of surface oxygen vacancies:

$$TOF = \frac{{kn}_{f , p - NP} {N}_{A }}{{ρ}_{vac}} \qquad ( S 7 )$$

Where: n_{f, p-NP}=amount of p-NP produced after reaction.

All error bars in figures were determined by calculating the standard error for repeated experiments.

S3. pH Adjustments of p-NPP Reaction and Phosphate Adsorption Solutions. Acidic and basic solutions were prepared to adjust the pH of the 10 mLp-NPP reaction solutions and phosphate adsorption solutions before the addition of CeO₂ nanocrystals. Solution pH was measured with a pH probe and confirmed with litmus paper tests before each reaction. The acidic pH modifier solution was prepared by diluting HNO3 with water to form a 0.073 M solution. The basic pH modifier solution was prepared by dissolved NaOH in water to produce a 0.250 M solution.

Although the present disclosure has been described with respect to one or more particular embodiments and/or examples, it will be understood that other embodiments and/or examples of the present disclosure may be made without departing from the scope of the present disclosure.

1. A method to catalyze a dephosphorylation reaction, comprising:
contacting a CeO₂ nanocrystal with a phosphate containing substrate to form a reaction mixture; and
incubating the reaction mixture for a period of time;
wherein after the period of time, the phosphate containing substrate is converted into a dephosphorylated product.

2. The method of claim 1, wherein the reaction mixture has a pH of 1-11.

3. The method of claim 2, wherein the reaction mixture has a pH of 8-11.

4. The method of claim 1, wherein the contacting a CeO₂ nanocrystal with a phosphate containing substrate to form a reaction mixture further comprises:
adjusting the reaction mixture before incubating to a first temperature; and
allowing the reaction mixture to remain at the first temperature for a first amount of time;
wherein after the first amount of time, the phosphate containing substrate is converted into a dephosphorylated product.

5. The method of claim 1, wherein the nanocrystal is chosen from a nanosphere, a nanooctahedron, a nanorod, and a nanorod.

6. The method of claim 4, wherein the first temperature is 3 to 95° C.

7. The method of claim 1, wherein the period of time is 1 second to 8 hours.

8. The method of claim 1, further comprising separating phosphorus or a phosphorus-containing compound from the dephosphorylated product.

9. The method of claim 1, further comprising:
centrifuging the reaction mixture after incubating and forming a supernatant and a pellet;
removing the supernatant;
suspending the pellet in a solution; and
sonicating the solution and pellet such that the pellet re-disperses into the solution.

10. The method of claim 1, wherein the phosphate containing substrate is present in a biomass, and/or a water effluent.

11. The method of claim 10, wherein the water effluent is a wastewater effluent.

12. An apparatus for separating phosphorus from a substrate, the apparatus comprising CeO₂ nanocrystals of claim 1.

13. The apparatus of claim 12, wherein the CeO₂ nanocrystals are present in a solution, or affixed to a substrate in the apparatus.

14. A method to recycle a used catalyst comprising:
suspending a used catalyst comprising CeO₂ nanocrystals in a solution; and
modulating the solution and the used catalyst such that the used catalyst re-disperses into the solution.

15. The method of claim 14, wherein the modulating comprises sonication.

16. The method of claim 10, further comprising incorporating the phosphorous and/or the phosphorus-containing compound from the dephosphorylated product into a distinct product.

17. A product formed by performing the method of claim 16.

18. A kit comprising CeO₂nanocrystals of claim 1.

19. The kit of claim 18, further comprising printed material including instructions for using the CeO₂ nanocrystals to separate phosphorus or a phosphorus containing compound from a substrate.
